(19) United States
(12) Patent Application Publication
(10) Pub. No.: US 2014/0251350 A1
(43) Pub. Date: Sep. 11, 2014

(54) MEDICAL IMMOBILIZATION DEVICES

(71) Applicant: Yechiel GOTFRIED, Kiryat Motzkin (IL)

(72) Inventor: Yechiel GOTFRIED, Kiryat Motzkin (IL)

(21) Appl. No.: 14/168,468

(22) Filed: Jan. 30, 2014

Related U.S. Application Data
(60) Provisional application No. 61/773,565, filed on Mar. 6, 2013.

(51) Int. Cl. A61F 5/37

(52) U.S. Cl. CPC A61F 5/3715
USPC 128/870

(57) ABSTRACT

An immobilization device includes a torso brace positioned around a subject's torso, a leg brace configured to be coupled around a respective leg of the subject, a connecting rod for each leg brace and at least one torso-brace joint assembly. Each joint assembly is coupled to the torso brace and a respective connecting rod and includes a torso-brace ball-and-socket joint and a torso-brace locking mechanism. The locking mechanism has an unlocked state in which pivoting of the connecting rod relative to the torso brace is possible, and a locked state in which such pivoting is prevented. A leg-brace joint assembly is coupled to each leg brace and its connecting rod, and includes a leg-brace ball-and-socket joint and a leg-brace locking mechanism having an unlocked state in which pivoting of the respective connecting rod relative to its leg brace is possible, and a locked state in which such pivoting is prevented.

### CROSS-REFERENCE TO RELATED APPLICATION

This application claims priority under 35 USC 119(e) of U.S. provisional patent application Ser. No. 61/773,565 filed Mar. 6, 2013, which is incorporated by reference herein.

### FIELD OF THE INVENTION

The present invention relates generally to orthopedic devices, and specifically to orthopedic immobilization devices.

### BACKGROUND OF THE INVENTION

Developmental dysplasia (dislocation) of the hip (DDH) is an abnormal formation of the hip joint in which the femoral head is not held firmly in the acetabulum.

DDH is often treated with surgery, during which the femur is manipulated into the proper position in the acetabulum. Afterwards, the patient, usually a child, is conventionally placed into a body cast (spica) to maintain the hip position, typically for a few weeks.

It is difficult to verify proper bone placement and to monitor treatment progress through the body cast, because the cast has low permeability to x-rays. The cast also prevents cleaning of body parts covered by the cast.

### SUMMARY OF THE INVENTION

Applications of the present invention provide immobilization devices for immobilizing a portion of a body of a human patient, such as the hip joint and the lower extremities, for treating conditions which require stable immobilization of the body portion in a precise position. The immobilization devices are easy, simple, and quick to assemble during an installation procedure. The components of the immobilization devices are typically transparent to x-rays and ultrasound, which enables verification of proper bone placement and monitoring of treatment progress. The immobilization devices may be used for treating various conditions, including but not limited to controlled reduction of developmental dysplasia (dislocation) of the hip (DDH), and immobilization of the hip for treating leg fractures, infection or correction of deformities.

One embodiment of an immobilization device in accordance with the invention comprises one or more braces, such as a torso brace and one or more limb braces. The immobilization device further comprises one or more connecting rods, which couple respective ones of the limb braces to the torso brace. In some embodiments, the limb braces comprise respective leg braces. In some embodiments, the immobilization device further comprises one or more torso-brace joint assemblies, which are coupled to the torso brace and respective connecting rods. Alternatively or additionally, the immobilization device further comprises one or more limb-brace joint assemblies, which are coupled to respective limb braces and removably coupled to respective connecting rods. The one or more torso-brace joint assemblies and the one or more limb-brace joint assemblies may be arranged in one or more joint series assemblies, which enable multi-directional movement of the one or more limbs braces with respect to the torso brace. The one or more braces, such as a torso brace and one or more limb braces, may comprise one or more members, so that the torso or limb brace comprises one, two or more members.

In some embodiments, each torso-brace joint assembly comprises (a) a torso-brace ball-and-socket joint, which comprises a torso-brace socket and a torso-brace ball fitted into the socket, and (b) a torso-brace locking mechanism. An end of the connecting rod is permanently or removably coupled to the torso-brace ball, such that torso-brace joint assembly is coupled to the connecting rod. Typically, the torso-brace locking mechanism comprises a ball-and-socket-joint locking mechanism. When in an unlocked state, the torso-brace locking mechanism allows pivoting of the connecting rod with respect to the torso-brace socket, and thus with respect to the torso brace, to which the torso-brace socket is coupled. When in a locked state, the torso-brace locking mechanism prevents pivoting of the connecting rod with respect to the torso-brace socket and the torso brace.

In some embodiments, the immobilization device further comprises one or more brace slide rods, each of which is fixed to the torso brace at one or more locations along the brace slide rod. In these applications, each torso-brace joint assembly comprises a brace-slide-rod interface, which is slidably coupled to one of the brace slide rods, so as to couple the torso-brace joint assembly to the torso brace via the brace slide rod. In some embodiments, the torso-brace locking mechanism comprises a brace-slide-rod locking mechanism, as well as the ball-and-socket-joint locking mechanism. When in an unlocked state, the torso-brace locking mechanism allows (i) sliding of the brace-slide-rod interface with respect to the brace slide rod, and (ii) pivoting of the connecting rod with respect to the torso-brace socket and the brace slide rod. When in a locked state, the torso-brace locking mechanism prevents (i) sliding of the brace-slide-rod interface with respect to the brace slide rod, and (ii) pivoting of the connecting rod with respect to the torso-brace socket and the brace slide rod.

Typically, a single mechanism simultaneously locks both the brace-slide-rod locking mechanism and the ball-and-socket-joint locking mechanism, and thus the torso-brace locking mechanism, and simultaneously unlocks both the brace-slide-rod locking mechanism and the ball-and-socket-joint locking mechanism, and thus the torso-brace locking mechanism.

### BRIEF DESCRIPTION OF THE DRAWINGS

The present invention will be more fully understood from the following detailed description of embodiments thereof, taken together with the drawings, in which:

FIGS. 1, 2A, 2B, 2C and 2D are schematic illustrations of an immobilization device, in accordance with respective applications of the present invention;

FIG. 3 is a schematic illustration of one configuration of a torso brace of the immobilization device, in accordance with an application of the present invention;

FIG. 4 is a schematic illustration of another configuration of the torso brace, in accordance with an application of the present invention;

FIGS. 5A, 5B, 5C, 6A, 6B and 6C are schematic illustrations of a torso-brace joint assembly of the immobilization device, in accordance with an application of the present invention;

FIG. 7 is a schematic illustration of another configuration of the torso brace, in accordance with an application of the present invention;

FIGS. 8A and 8B are schematic illustrations another configuration of the torso-brace joint assembly, in accordance with an application of the present invention;

FIGS. 8C, 8D and 8E are schematic illustrations of one limb-brace joint assembly of the immobilization device, in accordance with respective applications of the present invention;

FIGS. 9A and 9B are schematic illustrations of respective configurations of a joint series assembly of the immobilization device, in accordance with respective applications of the present invention; and

FIG. 10 is a schematic illustration of another immobilization device, in accordance with an application of the present invention.

### DETAILED DESCRIPTION OF THE INVENTION

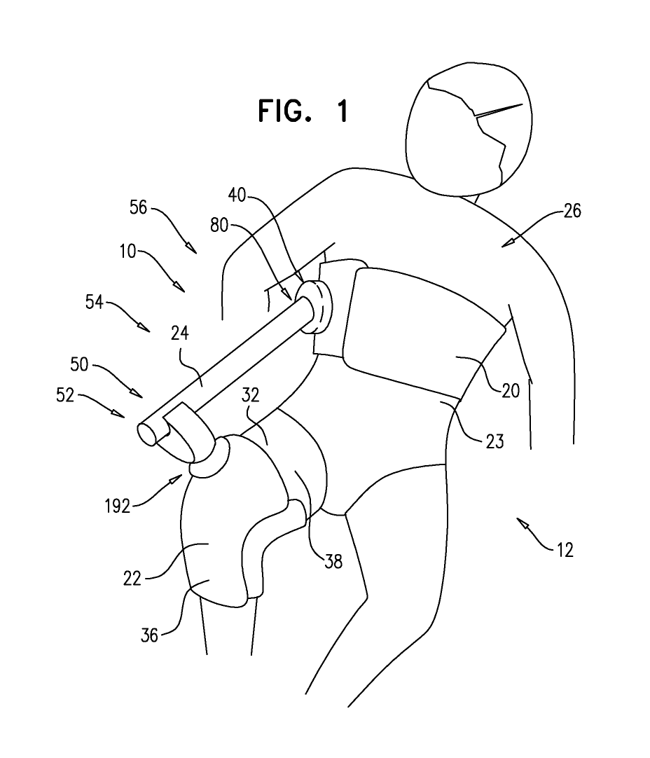

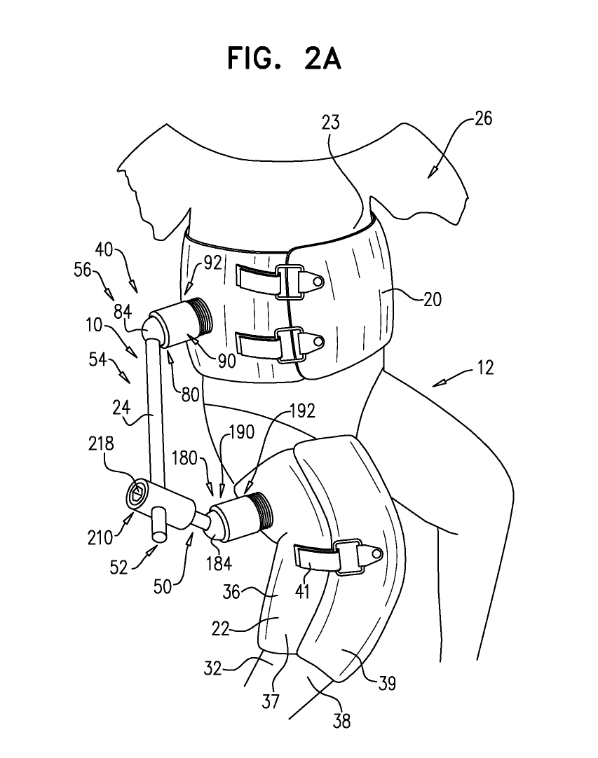

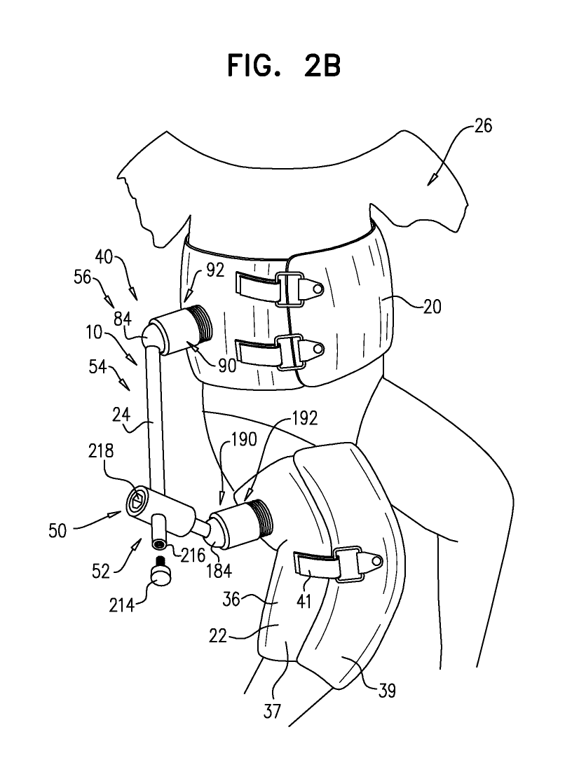

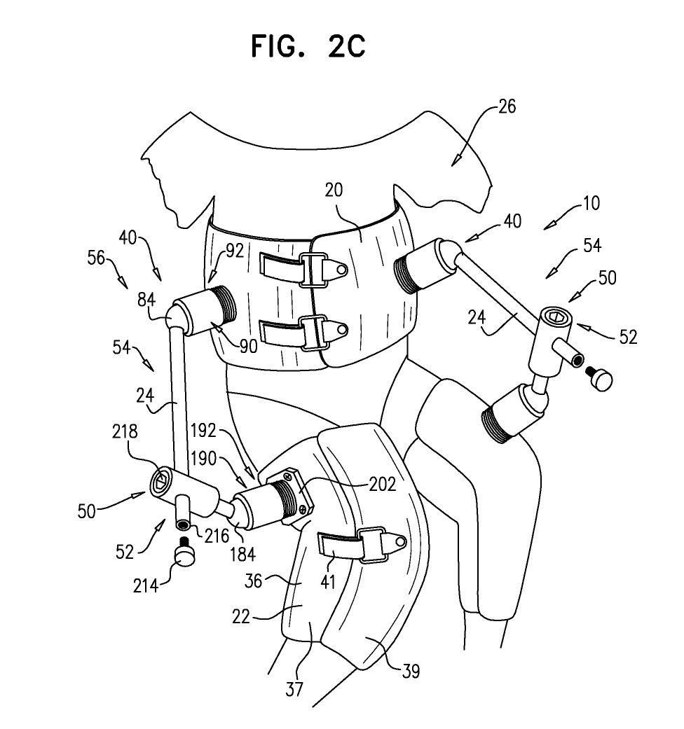

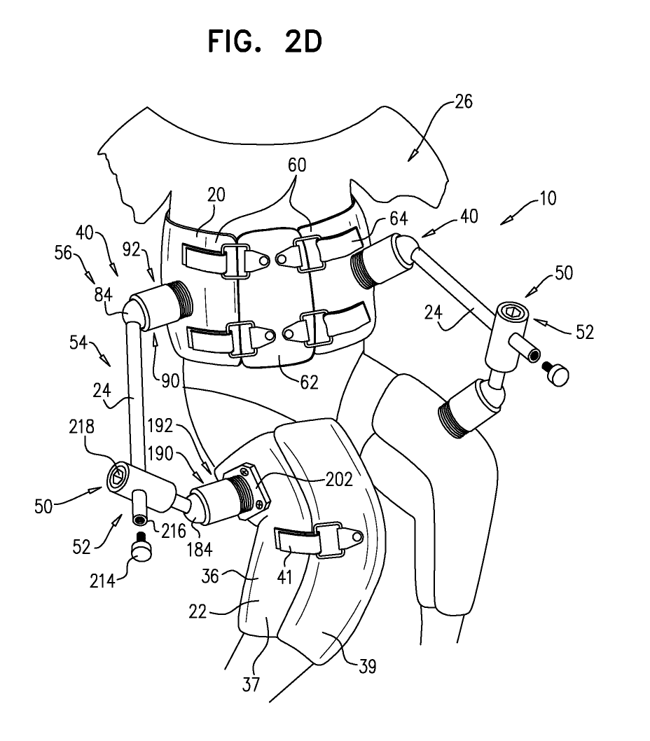

FIGS. 1 and 2A-2D are schematic illustrations of an immobilization device 10, in accordance with respective applications of the present invention. Immobilization device 10 comprises a brace system 12, which comprises one or more braces, such as a torso brace 20 and one or more limb braces 22, e.g., only two limb braces 22 as shown in FIGS. 2C and 2D. Immobilization device 10 further comprises one or more connecting rods 24, e.g., only two connecting rods 24, which couple respective ones of limb braces 22 to torso brace 20, as described in detail hereinbelow. For example, each connecting rod is attached at one end region to a respective limb brace 22 and at an opposite end region to the same torso brace 20. Torso brace 20 is configured to be securely coupled around a torso 23 of a subject 26, and to assume open and closed states. It is expected that the subject 26 will be human subject. Limb braces 22 are configured to be securely coupled around respective limbs 32 of the subject 26, and to assume open and closed states. In some embodiments, limb braces 22 comprise respective leg braces 36, e.g., only two leg braces 36, which are configured to be securely coupled around respective legs 38 of the subject 26. By way of illustration and not limitation, a single leg brace 36 and a single connecting rod 24 are shown in FIGS. 1, 2A and 2B, and exactly two leg braces 36 and exactly two connecting rods 24 are shown in FIGS. 2C and 2D. Alternatively, in some embodiments, limb braces 22 comprise respective leg braces 36, e.g., exactly two leg braces 36, which are configured to be securely coupled around respective legs 38 of the subject 26, and to be connected by a connecting rod 24.

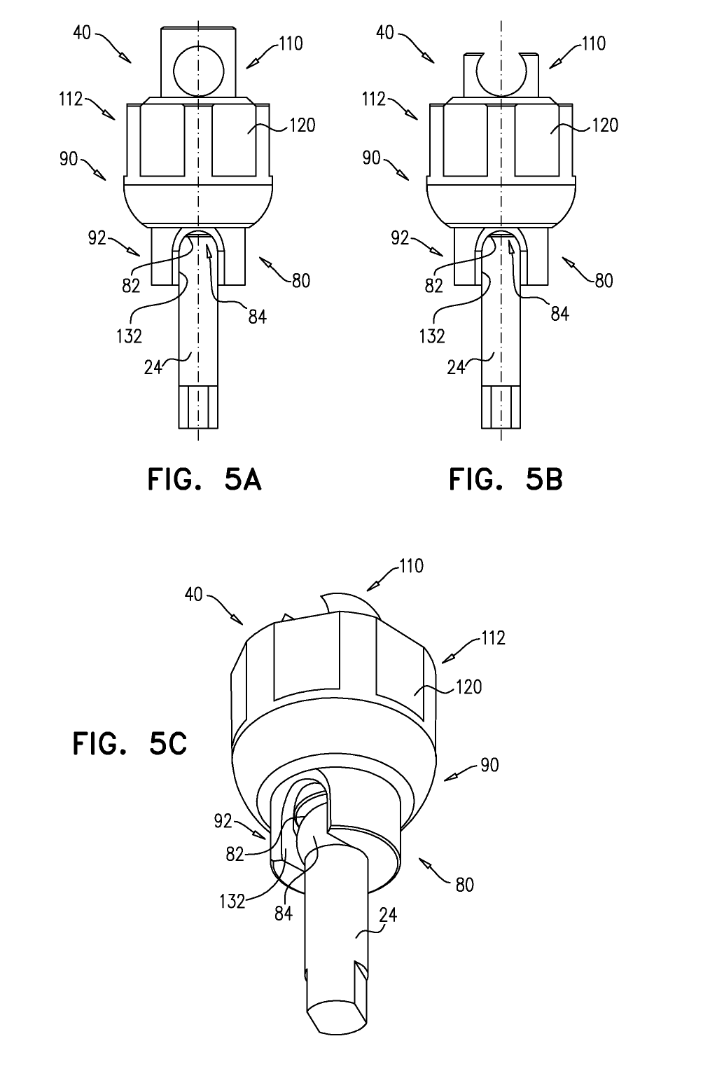

In some embodiments, immobilization device 10 further comprises one or more torso-brace joint assemblies 40, e.g., exactly two torso-brace joint assemblies 40, which are coupled to the torso brace 20 and respective connecting rods 24. Thus, each torso-brace joint assembly 40 is interposed between a respective connecting rod 40 and the torso brace 24. Several configurations of torso-brace joint assemblies 40 are described hereinbelow in detail with reference to FIGS. 5A-5C, 6A-6C, and 8A and 8B. By way of illustration and not limitation, a single torso-brace joint assembly 40 is shown in FIGS. 1, 2A-2D, 8A and 8B.

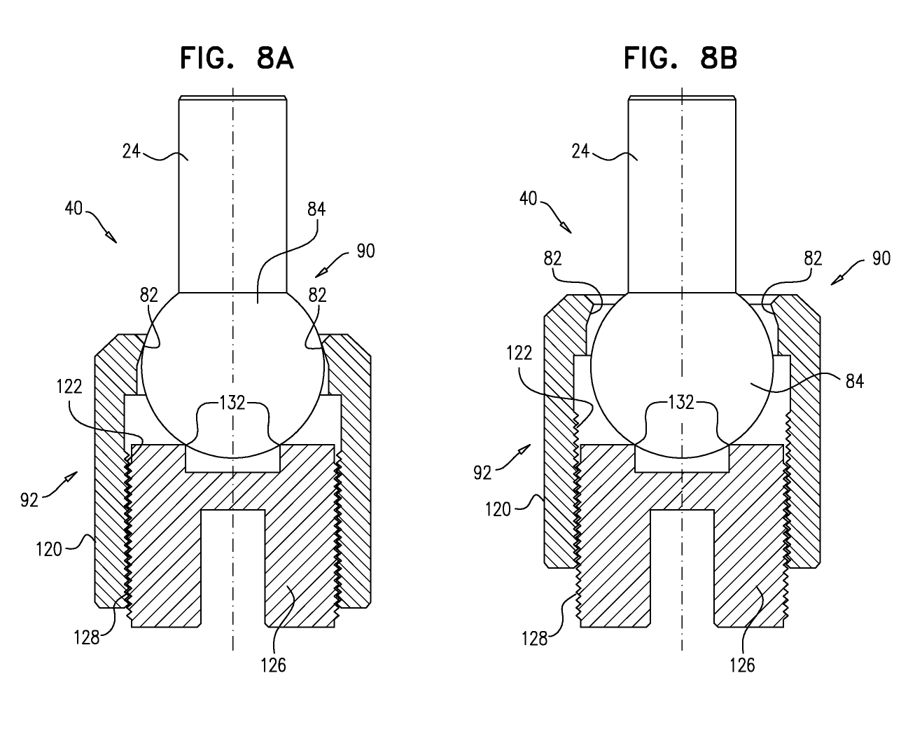

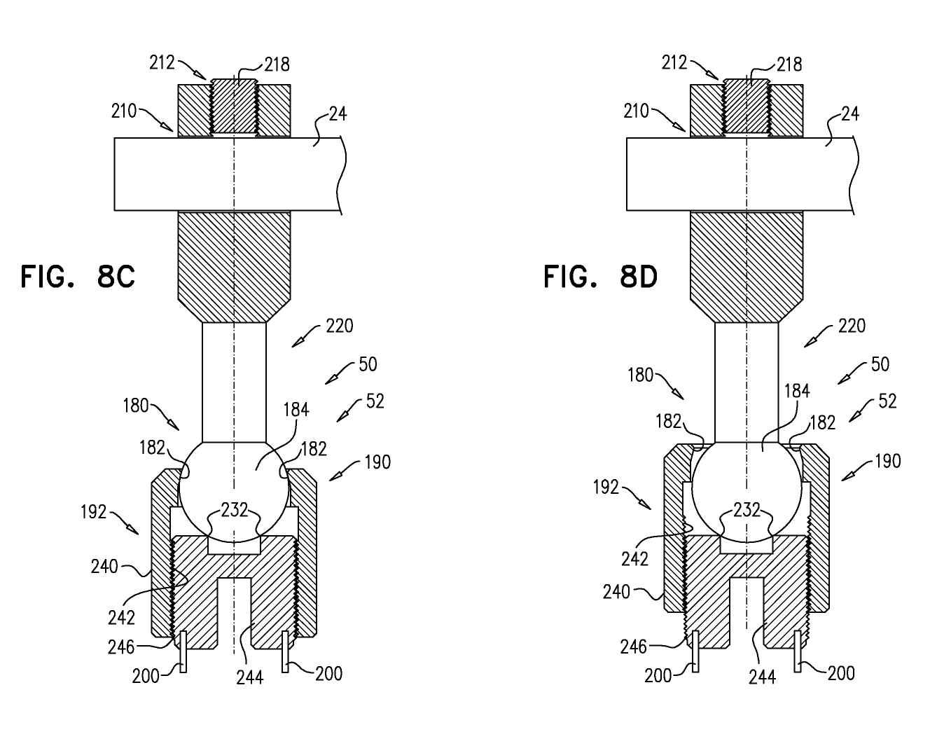

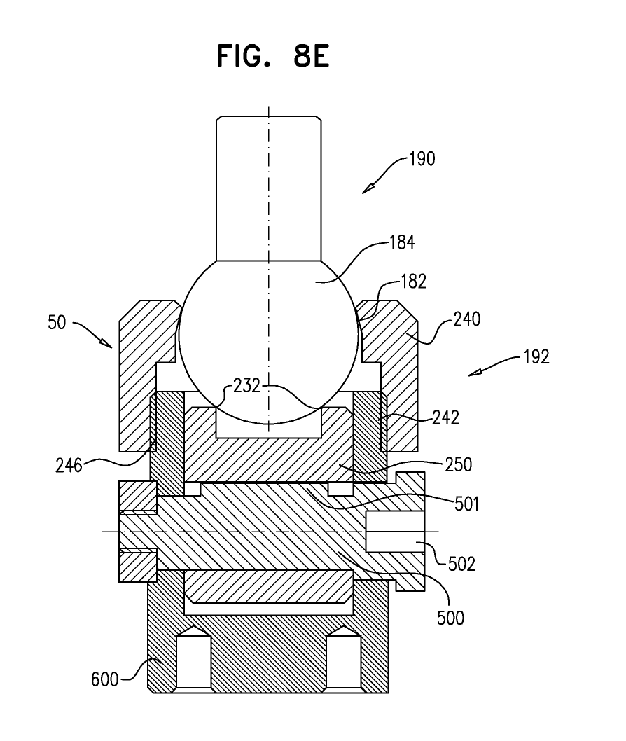

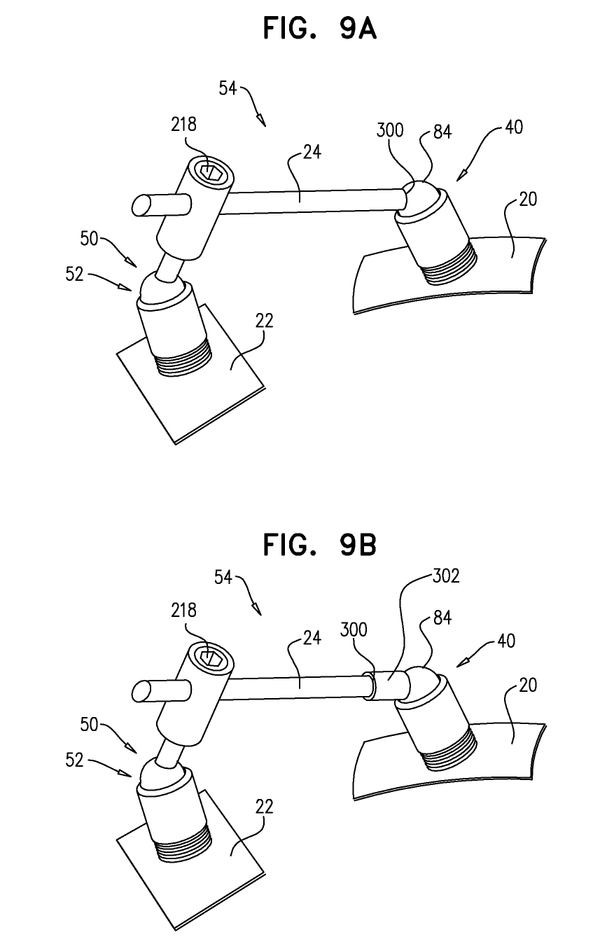

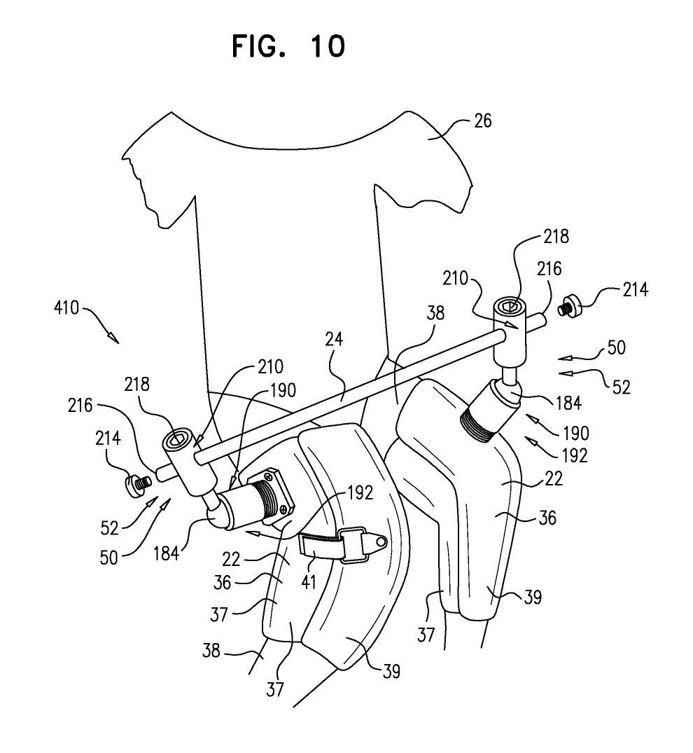

Alternatively or additionally, in some embodiments, immobilization device 10 further comprises one or more limb-brace joint assemblies 50, e.g., exactly two limb-brace joint assemblies 50, which are coupled to respective limb braces 22 and removably coupled to respective connecting rods 24. In the embodiments in which limb braces comprise respective leg braces 36, limb-brace joint assemblies 50 comprise respective leg-brace joint assemblies 52, e.g., exactly two respective leg-brace joint assemblies 52, which are coupled to respective leg braces 36 and respective connecting rods 24. Thus, each leg-brace joint assembly 52 is interposed between a respective connecting rod 40 and a respective leg brace 36. Several configurations of limb-brace joint assemblies 50 are described hereinbelow with reference to FIGS. 8C, 8D, 9A and 9B. By way of illustration and not limitation, a single leg-brace joint assembly 52 is shown in FIGS. 1, 2A and 2B, and exactly two leg-brace joint assemblies 52 (and/or two limb-brace joint assemblies 50) are shown in FIGS. 2C and 2D.

As shown in FIGS. 1 and 2A-2D, the one or more torso-brace joint assemblies 40 and the one or more limb-brace joint assemblies 50 are arranged in one or more joint series assemblies 54. Typically, each joint series assembly comprises one torso-brace joint assembly 40, one connecting rod 24, and one limb-brace joint assembly 50.

Typically, each connecting rod 24 comprises a metal, such as stainless steel, a plastic, a plastic composite, and/or a carbon composite.

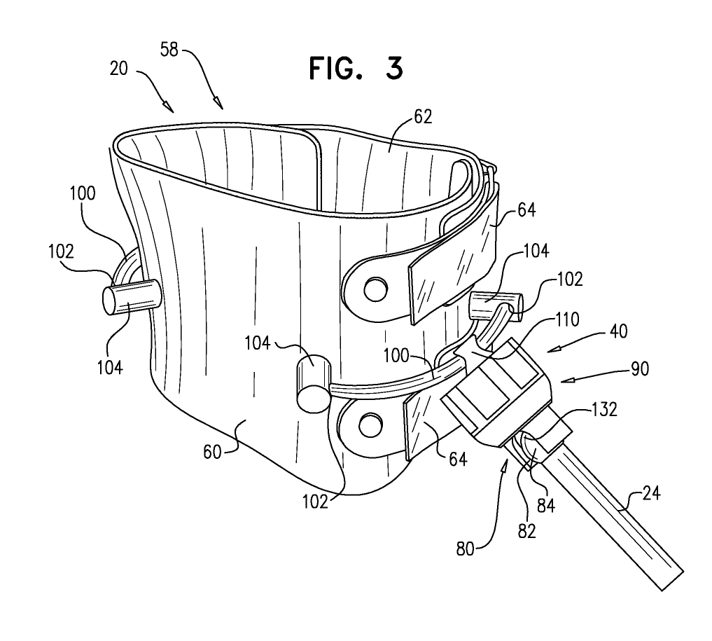

Reference is again made to FIG. 2D, and additionally to FIG. 3, which is a schematic illustration of one configuration of torso brace 20, in accordance with an application of the present invention. In the configurations shown in FIGS. 2D and 3, torso brace 20 comprises a two-piece torso brace 58. Two-piece brace 58 comprises at least separate first and second brace torso-brace sections 60 and 62, which are provided as separate sections that are configured to be securely coupled to one another around the torso of the human subject 36. For example, first brace section 60 may be a posterior brace section, and second brace section 62 may be an anterior brace section. First and second brace sections 60 and 62 may be coupled to each other by two sets of one or more securing elements 64. For example, securing elements 64 may comprise respective straps and buckles, snaps, and/or hook-and-loop fasteners such as VELCRO®. Typically, when coupled together, first and second brace sections 60 and 62 partially overlap each other, in order to allow the brace to be tightly fitted onto torsos of different sizes. Similarly, in some embodiments, each limb brace 22 (e.g., leg brace 36) comprises two limb-brace sections (e.g., two leg-brace sections) that are configured to be securely coupled to one another around the leg of the subject. It would be understood by those skilled in the art to which this invention pertains that the torso brace 20 may comprise more than two overlapping brace sections with appropriate securing elements to enable secure attachment to different-size torsos and similarly, that each limb brace 22 may comprise more than two overlapping brace sections with appropriate securing elements to enable secure attachment to different-size limbs.

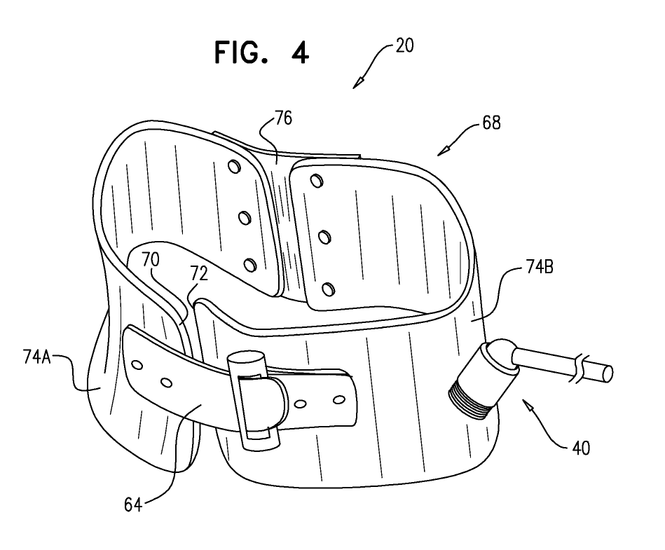

Reference is now made to FIG. 4, which is a schematic illustration of another configuration of torso brace 20, in accordance with an application of the present invention. In this configuration, torso brace 20 comprises a one-piece torso brace 68. One-piece torso brace 68 has first and second circumferential ends 70 and 72, which are configured to be securely coupled to one another around the torso of the human subject. First and second circumferential ends 70 and 72 may be coupled to each other by at least one set of one or more securing elements 64. For example, securing elements 64 may comprise respective straps and buckles, snaps, and/or hook-and-loop fasteners such as VELCRO®. In some embodiments, one-piece torso brace 68 comprises at least two sections 74A and 74B, and at least one hinge 76, such as a posterior hinge, which couples the sections 74A, 74B together such that the sections 74A, 74B can flex with respect to each other.

By way of illustration and not limitation, in FIGS. 1 and 2A-2C, torso brace 20 is shown as comprising a single-piece brace, and leg braces 36 are shown as comprising two-piece braces, for example, comprising first and second leg-brace sections 37 and 39, which may be coupled to each other by two sets of one or more securing elements 41.

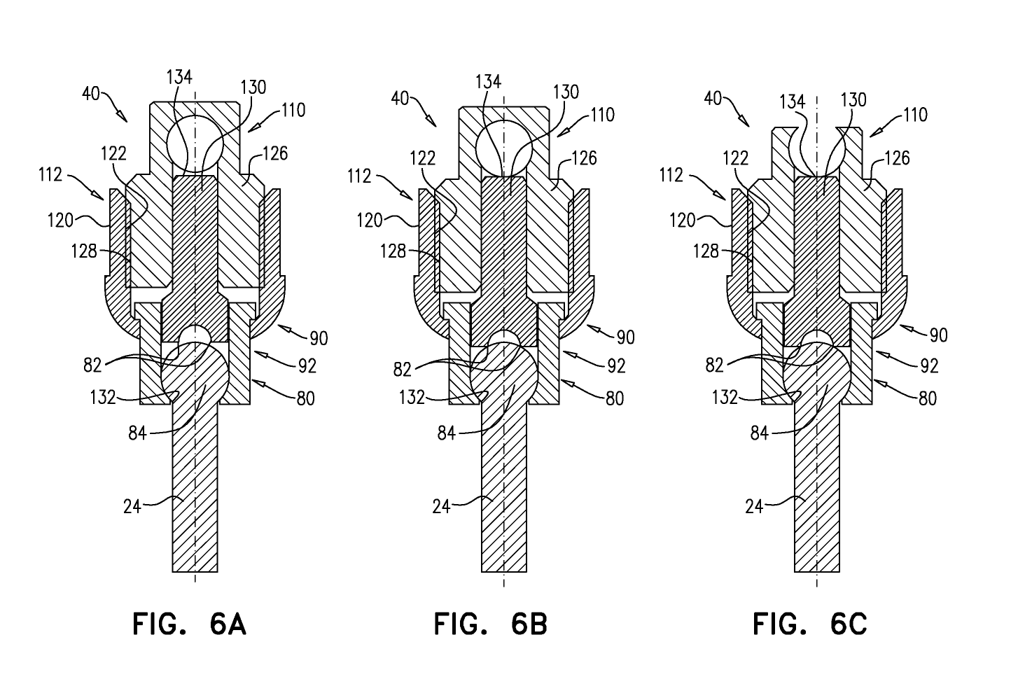

Reference is again made to FIGS. 2A-2D and 3, and additionally to FIGS. 5A-5C, 6A-6C, and 8A and 8B, which are schematic illustrations of one torso-brace joint assembly 40, in accordance with respective applications of the present invention. FIGS. 5A-5C and FIGS. 6A-6C are isometric and cross-sectional views, respectively, of two configurations of the torso-brace joint assembly 40, and FIGS. 8A and 8B are cross-sectional views of another configuration of the torso-brace joint assembly 40 in locked and unlocked states, respectively. As shown in FIGS. 5A-5C, 6A-6C, and 8A and 8B, each torso-brace joint assembly 40 comprises (a) a torso-brace ball-and-socket joint 80, which comprises a torso-brace socket 132 and a torso-brace ball 84 fitted into the socket 132, and (b) a torso-brace locking mechanism 90.

As is known in the mechanical arts, a ball-and-socket joint is a joint comprising a first segment shaped so as to define a bowl-shaped end (socket), and a second segment shaped so as to define a rounded end (ball) fitted into the bowl shape, allowing the second segment to move with respect to the first segment. It is noted that the ball is not necessarily entirely surrounded by the socket.

As mentioned above, each torso-brace joint assembly 40 (including torso-brace socket 132) is coupled to torso brace 20 and a connecting rod 24. In some embodiments, an end of connecting rod 24 is permanently coupled to torso-brace ball 84, such that torso-brace joint assembly 40 is fixedly coupled to connecting rod 24. Alternatively, the end of connecting rod 24 may be shaped so as to define torso-brace ball 84, such that torso-brace joint assembly 40 is coupled to connecting rod 24; in other words, torso-brace ball 84 may be an integral part of connecting rod 24. Further alternatively, the end of connecting rod 24 may be selectively or releasably coupled to the torso-brace ball 84, such that torso-brace joint assembly 40 is coupled to connecting rod 24.

Reference is still made to FIGS. 5A-5C, 6A-6C, and 8A and 8B. Typically, torso-brace locking mechanism 90 comprises a ball-and-socket-joint locking mechanism 92. When in an unlocked state (as shown in FIGS. 6A and 8B), torso-brace locking mechanism 90 allows pivoting of connecting rod 24 with respect to torso-brace socket 132 (as shown in FIGS. 6A and 8B), and thus with respect to torso brace 20, to which torso-brace socket 132 is coupled, as shown in FIGS. 2A-2D. Typically, when in the unlocked state, torso-brace locking mechanism 90 is entirely open and loose, thereby allowing the pivotal movement. When in a locked state (as shown in FIGS. 5A, 5B, 6B, 6C, and 8A), torso-brace locking mechanism 90 prevents the pivotal movement of connecting rod 24 with respect to torso-brace socket 132 and torso brace 20. In some embodiments, ball-and-socket-joint locking mechanism 92 is threaded.

Reference is made to FIGS. 3, 5A-5C, and 6A-6C. In some embodiments, such as shown in FIG. 3, immobilization device 10 further comprises one or more (e.g., exactly two) brace slide rods 100, each of which is fixed to torso brace 20 at one or more locations 102 along the brace slide rod 100. In the configuration shown in FIG. 3, locations 102 are at respective ends of the brace slide rods 100; alternatively, the locations are elsewhere along the brace slide rods 100, typically near the ends of the brace slide rods 100. In some embodiments, the brace slide rods 100 are fixed to the torso brace 20 via one or more short posts 104, as shown in FIG. 3. For some applications, at least a portion of each brace slide rod 100 is curved.

In the embodiments in which the immobilization device further comprises brace slide rods 100, each torso-brace joint assembly 40 comprises a brace-slide-rod interface 110, as shown in FIGS. 3, 5A-5C, and 6A-6C. Brace-slide-rod interface 110 is slidably coupled to one of brace slide rods 100, so as to couple the torso-brace joint assembly 40 to the torso brace 20 via the brace slide rod 100. In some embodiments, as shown in FIGS. 5A, 6A and 6B, brace-slide-rod interface 110 is shaped so as to entirely surround a portion of one of brace slide rods 100, such as when brace-slide-rod interface 110 comprises a relatively low-strength metal, such as aluminum. In other embodiments, such as shown in FIGS. 5B, 5C and 6C, brace-slide-rod interface 110 is shaped so as to partially surround a portion of one of brace slide rods 100, such as when brace-slide-rod interface 110 comprises a relatively strong metal, such as titanium.

In some embodiments, as shown in FIGS. 5A-5C and 6A-6C, torso-brace locking mechanism 90 comprises a brace-slide-rod locking mechanism 112, as well as ball-and-socket-joint locking mechanism 92. When in an unlocked state (as shown in FIG. 6A), torso-brace locking mechanism 90 allows (i) sliding of brace-slide-rod interface 110 with respect to brace slide rod 100 (i.e., brace-slide-rod locking mechanism 112 allows sliding), and (ii) pivoting of connecting rod 24 with respect to torso-brace socket 132 and brace slide rod 100 (i.e., ball-and-socket-joint locking mechanism 92 allows the pivotal movement). When in a locked state (as shown in FIGS. 5A, 5B, 6B, and 6C), torso-brace locking mechanism 90 prevents (i) sliding of brace-slide-rod interface 110 with respect to brace slide rod 100 (i.e., brace-slide-rod locking mechanism 112 prevents sliding), and (ii) pivoting of connecting rod 24 with respect to torso-brace socket 132 and brace slide rod 100 (i.e., ball-and-socket-joint locking mechanism 92 prevents the pivotal movement). In some embodiments, brace-slide-rod locking mechanism 112 is threaded, for example as described hereinbelow regarding cylindrical threaded chamber 122 and external threaded surface 128.

Typically, in embodiments in which torso-brace locking mechanism 90 comprises both brace-slide-rod locking mechanism 112 and ball-and-socket-joint locking mechanism 92, as shown in FIGS. 5A-5C and 6A-6C, a single mechanism simultaneously locks both brace-slide-rod locking mechanism 112 and ball-and-socket-joint locking mechanism 92, and thus torso-brace locking mechanism 90, and simultaneously unlocks both brace-slide-rod locking mechanism 112 and ball-and-socket-joint locking mechanism 92, and thus torso-brace locking mechanism 90.

In some embodiments, in order to provide such a single mechanism, as shown in FIGS. 5A-5C and 6A-6C, torso-brace locking mechanism 90 comprises:
a torso-brace locking external housing 120, which is shaped so as to define a cylindrical threaded, internal chamber 122;
an internal cylindrical member 126, which is an extension of brace-slide-rod interface 110, and which is shaped so as to define an external threaded surface 128 that is threadedly coupled to the cylindrical thread of chamber 122; and
an internal locking element 130, which is disposed within internal cylindrical member 126, and is shaped, on one end with a bearing surface 134 that functions to push and lock brace slide rod 100 when disposed inside brace-slide-rod interface 110, while the other end defines a torso-brace socket 132 that functions to push and lock connecting rod 24.

Rotation of torso-brace locking external housing 120 with respect to internal cylindrical member 126 transitions the torso-brace locking mechanism 90 between the unlocked state and the locked state thereof (one direction of rotation causing the unlocking while the opposite direction of rotation causes the locking). In some embodiments, in order to lock torso-brace locking mechanism 90, rotation of torso-brace locking external housing 120 with respect to internal cylindrical member 126 simultaneously:
moves locking surfaces 82 defined at one end of the internal locking element 130 toward the torso-brace socket 132, thereby pushing torso-brace ball 84 against the torso-brace socket 132 and preventing torso-brace ball 84 from pivoting with respect to the torso-brace socket 132, and
moves internal locking element 130 toward brace-slide-rod interface 110, thereby pushing the surface 134 of internal locking element 130 against brace slide rod 100 (when present in the brace-slide-rod interface 110), and preventing sliding of the brace slide rod 100 with respect to the brace-slide-rod interface 110 (for clarity of illustration, brace slide rod 100 is not shown in FIGS. 5A-5C, 6A, and 6B, but can be seen in FIG. 3).

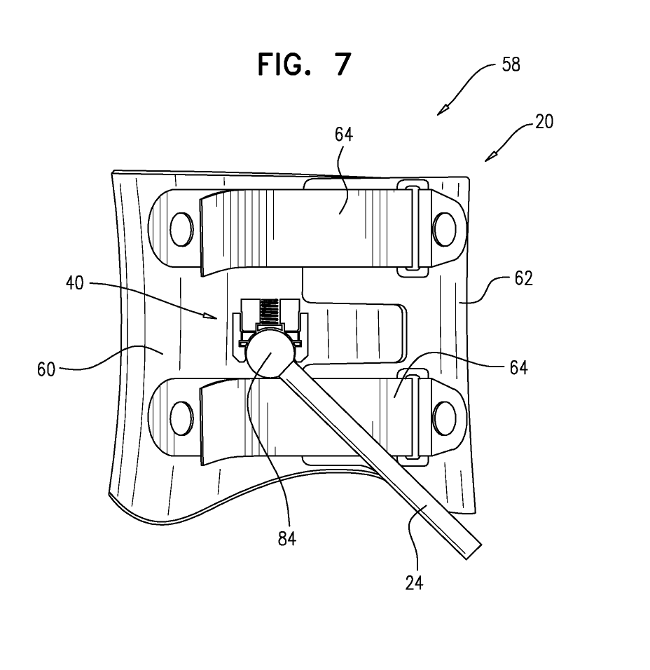

Reference is now made to FIGS. 1, 2A-2D, 7, 8A and 8B. FIG. 7 is a schematic illustration of another configuration of the torso brace 20, in accordance with an application of the present invention (in this configuration, torso brace 20 comprises a two-piece torso brace 20, such as described hereinabove with reference to FIG. 3). In these embodiments, immobilization device 10 does not comprise brace slide rods 100, and torso-brace locking mechanism 90 comprises only ball-and-socket-joint locking mechanism 92, and not brace-slide-rod locking mechanism 112, described hereinabove with reference to FIGS. 5A-5C and 6A-6C. FIGS. 8A and 8B show torso-brace locking mechanism 90 (and ball-and-socket-joint locking mechanism 92) in locked and unlocked states, respectively.

In some embodiments, as shown in FIGS. 8A and 8B, torso-brace locking mechanism 90 comprises:
torso-brace locking external housing 120, which is shaped so as to define a cylindrical threaded, internal chamber 122; and
internal cylindrical member 126, which is coupled to torso brace 20, and which is shaped so as to define external threaded surface 128 that is threadedly coupled to the cylindrical thread of chamber 122.

Rotation of torso-brace locking external housing 120 with respect to internal cylindrical member 126 transitions the torso-brace locking mechanism 90 between the unlocked state and the locked state thereof (one direction of rotation causing the unlocking while the opposite direction of rotation causes the locking). In some embodiments, in order to lock torso-brace locking mechanism 90, rotation of torso-brace locking external housing 120 with respect to internal cylindrical member 126 moves locking surfaces 82 toward torso-brace socket 132 (which is defined by internal cylindrical member 126), thereby pushing torso-brace ball against the torso-brace socket 132, and preventing torso-brace ball 84 from pivoting with respect to the torso-brace socket 132.

Reference is again made to FIGS. 2A-2D, and additionally to FIGS. 8C and 8D, which are schematic illustrations of one limb-brace joint assembly 50 in locked and unlocked states, respectively, in accordance with respective applications of the present invention. As mentioned above, each limb-brace joint assembly 50 is coupled to a respective limb brace 22 and a connecting rod 24. As mentioned above, in embodiments in which limb braces comprise respective leg braces 36, limb-brace joint assemblies 50 comprise respective leg-brace joint assemblies 52, which are coupled to respective leg braces and respective connecting rods 24. Although the following description refers to leg-brace applications, it applies equally well to more general limb-brace applications (e.g., arm-braces).

Each leg-brace joint assembly 52 comprises (a) a leg-brace ball-and-socket joint 180, which comprises a leg-brace socket 232 and a leg-brace ball 184 fitted into the socket, and (b) a leg-brace locking mechanism 190. Typically, each leg-brace joint assembly 52 (e.g., leg-brace socket 232 thereof) is fixed to a respective leg brace 36 (not shown in FIGS. 8C and 8D) by one or more coupling elements, such as pins 200 as shown in FIGS. 8C and 8D, and/or an intermediate interface plate 202, such as shown in FIGS. 2C and 2D. Each leg-brace joint assembly 52 further comprises a connecting-rod interface 210, which is slidably coupled to one of connecting rods 24.

Typically, leg-brace locking mechanism 190 comprises a ball-and-socket-joint locking mechanism 192. When in an unlocked state, as shown in FIG. 8D, leg-brace locking mechanism 190 allows pivoting of connecting-rod interface 210 (and thus connecting rod 24, when inserted in interface 210) with respect to leg-brace socket 232, and thus with respect to leg brace 36, to which leg-brace socket 232 is coupled. When in a locked state, as shown in FIG. 8C, leg-brace locking mechanism 190 prevents pivoting of connecting-rod interface 210 (and thus connecting rod 24) with respect to leg-brace socket 232 and leg brace 36. For some applications, ball-and-socket-joint locking mechanism 192 is threaded.

In some embodiments, as shown in FIGS. 8C and 8D (and FIGS. 2A-2D), connecting-rod interface 210 is shaped so as to entirely surround a portion of one of connecting rods 24. This configuration may reduce the possibility of the connecting rod 24 separating from the connecting-rod interface at stages of the application procedure after the connecting rod 24 has been connected to the interface 210. In some of these applications, such as shown in FIGS. 2A-2D, 8C and 8D, a screw 218 is inserted at the outer end of connecting-rod interface 210, and used to lock connecting rod 24 inside connecting-rod interface 210. Additionally, in some of these embodiments, an end cap 214, such as shown in FIGS. 2B-2D, is provided for each connecting rod 24. Each end cap 214 has a diameter greater than the diameter of connecting rod 24 and the hole of connecting-rod interface 210, and is used to prevent connecting rod 24 from coming out of connecting-rod interface 210 after assembly.

In some embodiments, joint assembly 52 comprises a linking member 220, which is shaped so as to define leg-brace ball 184 at a first end thereof, and connecting-rod interface 210 near another end thereof (see FIGS. 8C and 8D). Typically, connecting-rod interface 210 is shaped so as to define an opening that is sized to slidingly accept connecting rod 24.

In the embodiments as shown in FIGS. 8C-8D, leg-brace ball-and-socket joint 180 comprises:
a ball-and-socket external housing 240, which is shaped so as to define a cylindrical threaded, internal chamber 242; and
an internal cylindrical member 244, which is coupled to leg brace 36, and which is shaped so as to define an external threaded surface 246 that is threadedly coupled to the cylindrical thread of chamber 242.

Rotation of the ball-and-socket external housing 240 with respect to internal cylindrical member 244 transitions ball-and-socket-joint locking mechanism 192 between the ball-and-socket-unlocked state and the ball-and-socket-locked state (one direction of rotation causing the unlocking while the opposite direction of rotation causes the locking). Typically, rotation of the ball-and-socket external housing 240 in one direction moves ball-and-socket external housing 240 toward coupling elements 200 and the brace, tightening locking surfaces 182 defined by external housing 240 against leg-brace ball 184, which pushes the leg-brace ball 184 against leg-brace socket 232. Coupling elements 200, and/or intermediate interface plate 202, such as shown in FIGS. 2C and 2D, if provided, prevent rotation of internal cylindrical member 244 during rotation of ball-and-socket external housing 240.

In the embodiment shown in FIG. 8E, leg-brace ball-and-socket joint 190 comprises:
a ball-and-socket external housing 240, which is shaped so as to define a cylindrical threaded, internal chamber 242; and
an internal base member 600, which is coupled to leg brace 36, and which is shaped so as to define an external threaded surface 246 that is threadedly coupled to the cylindrical thread of chamber 242,
wherein the internal base member also includes an internal piston 250 comprising on its upper end, a socket 232 adapted to move up and down along the longitudinal axis of the internal base member 600, the up and down movement being activated by an eccentric bolt 500 located inside the internal piston 250, whereby turning the eccentric bolt 500 via a recess 502 moves the internal piston 250 upward thereby locking the socket 232 to the ball 184.

Rotation of the ball-and-socket external housing 240 with respect to internal base member 600 in one direction of rotation, moves ball-and-socket external housing 240 toward internal base member 600, and the brace, as well as toward socket 232 of internal piston 250 to a maximal proximity, at this stage upwards movement of socket 232 of internal piston 250 is activated by the eccentric bolt 500. That is, turning the eccentric bolt 500 via the recess 502 causes the internal piston 250 to move upwards, thereby locking the socket 232 to the ball 184.

Reference is again made to FIGS. 2B-2D. In embodiments in which connecting-rod interface 210 is shaped so as to entirely surround a portion of one of connecting rods 24, as shown in FIGS. 2B-2D, 8C and 8D, end cap 214, shown in FIGS. 2B-2D, is provided for each connecting rod 24. The end cap 214 is configured to be coupled to an end 216 of the connecting rod, so as to prevent connecting rod 24 from sliding out of connecting-rod interface 210. To this end, an outer diameter of the end cap 214 is greater than that of connecting rod 24 and the hole through connecting-rod interface 210. For some applications, the end cap 214 is coupled to the connecting rod 24, such as by screwing, during the installation procedure after sliding the connecting rod 24 through the connecting-rod interface 210. Alternatively, in embodiments in which immobilization device 10 is provided with the connecting rod 24 pre-connected to the connecting-rod interface 210, the end cap 214 is provided pre-installed on the connecting rod 24. Typically, the end cap 214 is shaped so as to define a screw thread.

In some embodiments, such as shown in FIGS. 2A-2D, 8C and 8D, leg-brace locking mechanism 190 comprises a connecting-rod locking mechanism 212 (labeled in FIGS. 8C and 8D), as well as ball-and-socket-joint locking mechanism 192. When in a rod-unlocked state (as shown in FIGS. 8C and 8D), connecting-rod locking mechanism 212 allows sliding of connecting-rod interface 210 with respect to the connecting rod 24. When in a rod-locked state (as shown in FIGS. 2A-2D), connecting-rod locking mechanism 212 prevents sliding of connecting-rod interface 210 with respect to the connecting rod 24. Typically, connecting-rod locking mechanism 212 and ball-and-socket-joint locking mechanism 192 are configured to be separately locked and unlocked. For some applications, connecting-rod locking mechanism 212 is threaded. In some embodiments, connecting-rod locking mechanism 212 comprises screw 218, which is inserted at the outer end of connecting-rod interface 210, and used to lock connecting rod 24 inside connecting-rod interface 210. For example, screw 218 may comprise a M5 screw and/or may be headless.

In some embodiments, torso-brace joint assemblies 40 are identical in design to limb-brace joint assemblies 50 (e.g., leg-brace joint assemblies 52), other than in the manner in which they interface with connecting rod 24. For other applications, torso-brace joint assemblies 40 are different in design from limb-brace joint assemblies 50 (e.g., leg-brace joint assemblies 52).

Reference is again made to FIGS. 2A-2D, and additionally to FIGS. 9A, 9B and 10, which are schematic illustrations of another configuration of one limb-brace joint assembly 50, in accordance with respective applications of the present invention. As mentioned above, each limb-brace joint assembly 50 is coupled to a respective limb brace 22 and a connecting rod 24. As mentioned above, for applications in which limb braces 22 comprise respective leg braces 36, limb-brace joint assemblies 50 comprise respective leg-brace joint assemblies 52, which are coupled to respective leg braces and respective connecting rods 24. Although the following description refers to leg-brace applications, it applies equally well to more general limb-brace applications (e.g., arm-braces).

As mentioned above, and as shown in FIGS. 9A and 10, each leg-brace joint assembly 52 comprises (a) leg-brace ball-and-socket joint 180, which comprises leg-brace socket 232 and leg-brace ball 184 fitted into the socket, and (b) leg-brace locking mechanism 190. Typically, each leg-brace joint assembly 52 (e.g., leg-brace socket 232 thereof) is fixed to a respective leg brace 36 (not shown in FIGS. 9A, 9B and 10) by one or more coupling elements, such as pins 200, screws, or both, and/or an intermediate interface plate 202.

Typically, leg-brace locking mechanism 190 comprises ball-and-socket-joint locking mechanism 192. When in an unlocked state (as shown in FIG. 8D, which is described hereinabove), ball-and-socket-joint locking mechanism 192 allows pivoting of connecting-rod interface 210 (and thus connecting rod 24, when inserted in interface 210) with respect to leg-brace socket 232, and thus with respect to leg brace 36, to which leg-brace socket 232 is coupled. When in a locked state (as shown in FIGS. 9A and 10), ball-and-socket-joint locking mechanism 192 prevents pivoting of connecting-rod interface 210 (and thus connecting rod 24) with respect to leg-brace socket 232 and leg brace 36. For some applications, ball-and-socket-joint locking mechanism 192 is threaded.

In some embodiments, each leg-brace joint assembly 52 comprises connecting-rod interface 210, which is slidably coupled to one of the connecting rods 24.

Alternatively or additionally, as described hereinabove with reference to FIGS. 8C-8E, leg-brace ball-and-socket joint 180 comprises:
ball-and-socket external housing 240, which is shaped so as to define cylindrical threaded, internal chamber 242; and
internal cylindrical member 244, which is coupled to leg brace 36, and which is shaped so as to define external threaded surface 246 that is threadedly coupled to the cylindrical thread of chamber 242.

Rotation of the ball-and-socket external housing 240 with respect to internal cylindrical member 244 transitions ball-and-socket-joint locking mechanism 192 between the ball-and-socket-unlocked state and the ball-and-socket-locked state (one direction of rotation causing the unlocking while the opposite direction of rotation causes the locking). Typically, rotation of the ball-and-socket external housing 240 moves ball-and-socket external housing 240 toward coupling elements 200 and the brace, tightening locking surfaces 182 defined by external housing 240 against leg-brace ball 184, which pushes the ball against leg-brace socket 232. Coupling elements, such as pins 200, or intermediate interface plate 202, if provided, prevent rotation of internal cylindrical member 244 during rotation of ball-and-socket external housing 240. Alternatively, intermediate interface plate 202 may consist not of a separate part, but rather may be an integral part of internal cylindrical member 244.

Typically, during locking of leg-brace locking mechanism 190 during installation of immobilization device 10, the physician or another assisting person first locks connecting-rod locking mechanism 212, and subsequently locks ball-and-socket-joint locking mechanism 192. Alternatively, the physician or another assisting person performs the locking in the reverse order.

Reference is again made to FIGS. 1 and 2A-2D. Typically, the one or more torso-brace joint assemblies 40 and the one or more limb-brace joint assemblies 50 are configured, when coupled together by one or more respective connecting rods 24 and coupled to the one or more braces, to provide a frame 56 that is configured to assume:
a frame-unlocked state when (a) ball-and-socket-joint locking mechanism 92 is in a first ball-and-socket-unlocked state, (b) brace-slide-rod locking mechanism 112, if provided, is in a first rod-unlocked state, (c) ball-and-socket-joint locking mechanism 192 is in a second ball-and-socket-unlocked state, and (d) connecting-rod locking mechanism 212 (shown in FIGS. 9A and 10), if provided, is in a second rod-unlocked state, in which frame-unlocked state each of the braces 20 and 22 can be rotated and tilted to a desired anatomical position, and
a frame-locked state when (a) ball-and-socket-joint locking mechanism 92 is in a first ball-and-socket-locked state, (b) brace-slide-rod locking mechanism 112, if provided, is in a first rod-locked state, (c) ball-and-socket-joint locking mechanism 192 is in a second ball-and-socket-locked state, and (d) connecting-rod locking mechanism 212 (shown in FIGS. 9A and 10), if provided, is in a second rod-locked state, and in the frame-locked state, the one or more braces and 22 are locked in the desired anatomical position.

Reference is now made to FIGS. 9A and 9B, which are schematic illustrations of respective configurations of one joint series assembly 54, in accordance with respective applications of the present invention. As mentioned above, each torso-brace joint assembly 40 is coupled to the torso brace 20 and a respective connecting rod 24.

In the configuration shown in FIG. 9A:
an end 300 of connecting rod 24 is permanently coupled to the torso-brace ball 84, such that torso-brace joint assembly 40 is fixedly coupled to connecting rod 24; or
end 300 of connecting rod 24 is shaped so as to define torso-brace ball 84, such that torso-brace joint assembly 40 is coupled to connecting rod 24; in other words, torso-brace ball 84 is an integral part of connecting rod 24.

In the configuration shown in FIG. 9B, end 300 of connecting rod 24 is coupleable to torso-brace ball 84, such that torso-brace joint assembly 40 is coupled to connecting rod 24. For example, end 300 of connecting rod is coupleable to torso-brace ball 84 via a coupling interface 302 that is permanently coupled to torso-brace ball 84, or defined by torso-brace ball 84. Typically, an end 300 is screwed into coupling interface 302, or directly into torso-brace ball 84.

In some embodiments, a kit is provided that comprises a plurality of connecting rods 24 having different respective lengths. The physician or an assistance person selects a rod having an appropriate length, based on the size of the subject.

In some applications of the present invention, immobilization device 10 is applied to the subject 26 after performing a closed or open orthopedic surgical procedure which requires subsequent immobilization of certain anatomical body portions in an exact anatomical position. An immobilization device 10 of appropriate size is selected, and components of the immobilization device needed for certain anatomical body portions are chosen as well. These components may include, for example, a torso brace 20 and one or more limb braces 22, e.g., exactly one limb brace 22 or exactly two limb braces 22.

The selected braces are placed into their respective open states, and are placed around their respective anatomical body portions. The braces are typically closed at this stage of the procedure by using securing elements 64. If torso-brace sections 60 and 62 and/or limb-brace sections 37 and 39 are being used, they are typically closed at this stage using securing elements 41.

In some embodiments, at this stage of the procedure one or more torso-brace joint assemblies 40 and one or more limb-brace joint assemblies 50, as well as one or more connecting rods 24, are assembled into one or more joint series assemblies 54, each of which connects respective pairs of braces. Alternatively, all or a portion of the assembly is formed before placing the braces around their respective anatomical body portions. All of the locking mechanisms are unlocked, in order to allow maximum flexibility of the components of the immobilization device.

If the braces have not already been closed, the braces are now closed around their respective anatomical body portions while the entire series of locking mechanisms is opened to the maximum, providing a maximal loosened state.

All of the anatomical body portions are positioned in their respective desired exact anatomical positions of immobilization. Such positioning may be performed using medical visualization imaging, such as x-ray or ultrasound. The required positions may be verified using imaging. If the positions are satisfactory, all of the locking mechanisms are transitioned to their respective locked states, completing the procedure.

Optionally, the anterior brace section of the torso brace may be removed for ultrasound examination.

Reference is now made to FIG. 10, which is a schematic illustration of an abduction immobilization device 410, in accordance with an application of the present invention. Except as described below, immobilization device 410 is generally similar to immobilization device 10, described hereinabove with reference to FIGS. 1-9B, and may incorporate any of the features of immobilization device 10.

Abduction immobilization device 410 device comprises two limb braces 22, which, for some applications, comprise respective leg braces 36, e.g., exactly two leg braces 36, which are configured to be securely coupled around respective legs 38 of the subject, and to be connected by a single connecting rod 24. Unlike immobilization device 10, abduction immobilization device 410 typically does not comprise a torso brace 20. Abduction immobilization device 410 is used to immobilize the two limbs, e.g., two legs, with respect to each other. For some applications, immobilization device 10 is applied and used during a first post-operative period (such as about six weeks), and thereafter immobilization device 10 is removed and abduction immobilization device 410 is applied used during a second post-operative period.

It is to be understood that all of the features described herein regarding torso brace 20 and torso-brace joint assembly 40 may alternatively or additionally be implemented in one or more of limb braces 22 and/or in one or more of limb-brace joint assemblies 50, and vice versa. For example, torso-brace joint assembly 40 may be removably coupled to one or more connecting rods 24, and limb-brace joint assemblies 50 may be coupled to respective ones of the connecting rods.

Described above are various configurations of an immobilization device in accordance with the invention. The different parts of the device may be used in numerous, different combinations with one another such as described above or otherwise. Some of these combinations are as follows.

In embodiments of the invention that include limb braces, the limb braces may comprise respective leg braces. For these embodiments, the limb-brace joint assemblies comprise respective leg-brace joint assemblies. Each leg-brace joint assembly comprises: (a) a leg-brace ball-and-socket joint, which comprises a leg-brace socket and a leg-brace ball fitted into the socket, and (b) a leg-brace locking mechanism. Typically, each leg-brace joint assembly is fixed to a respective leg brace by one or more coupling elements, such as pins, screws, or both; these can be coupled directly to the limb braces or with an interface plate in between to increase the contact area and hence stability of the coupling.

In some embodiments, each of the leg-brace joint assemblies comprises a connecting-rod interface, which is slidably coupled to one of the connecting rods. In some embodiments, the connecting-rod interface is shaped so as to entirely surround a portion of one of the connecting rods. In other embodiments, the connecting-rod interface is shaped so as to partially surround a portion of one of the connecting rods. The latter configuration may facilitate easier coupling of the connecting rod to the connecting-rod interface during an application procedure, while the former configuration may reduce the possibility of the connecting rod separating from the connecting-rod interface at stages of the application procedure after the connecting rod has been connected to the interface.

Typically, the leg-brace locking mechanism comprises a ball-and-socket-joint locking mechanism. When in an unlocked state, the leg-brace locking mechanism allows pivoting of the connecting-rod interface (and thus the connecting rod, when inserted in the interface) with respect to the leg-brace socket, and thus with respect to the leg brace, to which the leg-brace socket is coupled. When in a locked state, the leg-brace locking mechanism prevents pivoting of the connecting-rod interface (and thus the connecting rod) with respect to the leg-brace socket and the leg brace.

In some embodiments, the leg-brace locking mechanism comprises a connecting-rod locking mechanism, as well as the ball-and-socket-joint locking mechanism. When in a rod-unlocked state, the connecting-rod locking mechanism allows sliding of the connecting-rod interface with respect to the connecting rod. When in a rod-locked state, the connecting-rod locking mechanism prevents sliding of the connecting-rod interface with respect to the connecting rod. Typically, the connecting-rod locking mechanism and ball-and-socket-joint locking mechanism are configured to be separately locked and unlocked. In some embodiments, the joint assembly comprises a linking member, which is shaped so as to define the leg-brace ball at a first end thereof, and a post at a second end thereof, which post is coupled to the connecting-rod interface.

Typically, the one or more torso-brace joint assemblies and the one or more limb-brace joint assemblies are configured, when coupled together by one or more respective connecting rods and coupled to the one or more braces, to provide a frame that is configured to assume:
a frame-unlocked state when (a) the ball-and-socket-joint locking mechanism is in a first ball-and-socket-unlocked state, (b) the brace-slide-rod locking mechanism, if provided, is in a first rod-unlocked state, (c) the ball-and-socket-joint locking mechanism is in a second ball-and-socket-unlocked state, and (d) the connecting-rod locking mechanism is in a second rod-unlocked state, in which frame-unlocked state each of the braces can be rotated and tilted to a desired anatomical position, and
a frame-locked state when (a) the ball-and-socket-joint locking mechanism is in a first ball-and-socket-locked state, (b) the brace-slide-rod locking mechanism, if provided, is in a first rod-locked state, (c) the ball-and-socket-joint locking mechanism is in a second ball-and-socket-locked state, and (d) the connecting-rod locking mechanism is in a second rod-locked state, in which frame-locked state the one or more braces are locked in the desired anatomical position.

In some embodiments of the present invention, the immobilization device is applied to the subject after performing a closed or open orthopedic surgical procedure which requires subsequent immobilization of certain anatomical body portions in an exact anatomical position. An immobilization device of appropriate size is prepared in advance or selected, and components of the immobilization device needed for certain anatomical body portions are chosen as well.

These components may include, for example, a torso brace and one or more limb braces, e.g., exactly one limb brace or exactly two limb braces.

The selected braces are placed into their respective open states, and are placed around their respective anatomical body portions. The braces are typically left open at this stage of the procedure.

In some embodiments, at this stage of the procedure, one or more torso-brace joint assemblies and one or more limb-brace joint assemblies, as well as one or more connecting rods, are assembled into one or more joint series assemblies, each of which connects respective pairs of braces. Alternatively, all or a portion of the assembly is formed before placing the braces around their respective anatomical body portions. All of the locking mechanisms are unlocked, in order to allow maximum flexibility of the components of the immobilization device.

Typically, the braces are now closed around their respective anatomical body portions.

All of the anatomical body portions are positioned in their respective desired exact anatomical positions of immobilization. Such positioning may be performed using medical visualization imaging, such as x-ray or ultrasound. The required positions may be verified using imaging. If the positions are satisfactory, all of the locking mechanisms are transitioned to their respective locked states, completing the procedure.

There is therefore provided, in accordance with an embodiment of the present invention, an immobilization system which includes:

a torso brace, which is configured to be securely coupled around a torso of a human subject;

a leg brace, which is configured to be securely coupled around a leg of the subject;

a connecting rod;

a torso-brace joint assembly, which is coupled to the torso brace and the connecting rod, and which includes (a) a torso-brace ball-and-socket joint including a torso-brace socket and a torso-brace ball fitted into the socket, and (b) a torso-brace locking mechanism, which torso-brace locking mechanism (i) when in an unlocked state, allows pivoting of the connecting rod with respect to the torso brace, and (ii) when in a locked state, prevents pivoting of the connecting rod with respect to the torso brace; and

a leg-brace joint assembly, which is coupled to the leg brace and the connecting rod, and which includes (a) a leg-brace ball-and-socket joint including a leg-brace socket and a leg-brace ball fitted into the socket, and (b) a leg-brace locking mechanism, which leg-brace locking mechanism (i) when in an unlocked state, allows pivoting of the connecting rod with respect to the leg brace, and (ii) when in a locked state, prevents pivoting of the connecting rod with respect to the leg brace.

In some embodiments, an end of the connecting rod is permanently coupled to the torso-brace ball, such that the torso-brace joint assembly is coupled to the connecting rod. Alternatively, in some embodiments, an end of the connecting rod is configured to be selectively coupled to the torso-brace ball, such that the torso-brace joint assembly can be coupled to the connecting rod. Further alternatively, in some embodiments, an end of the connecting rod is shaped so as to define the torso-brace ball, such that the torso-brace joint assembly is coupled to the connecting rod.

In some embodiments, an end of the connecting rod is permanently coupled to the leg-brace ball, such that the leg-brace joint assembly is coupled to the connecting rod. Alternatively, in some embodiments, an end of the connecting rod is configured to be selectively coupled to the leg-brace ball, such that the leg-brace joint assembly can be coupled to the connecting rod. Further alternatively, in some embodiments, an end of the connecting rod is shaped so as to define the leg-brace ball, such that the leg-brace joint assembly is coupled to the connecting rod.

In some embodiments, the leg-brace socket is coupled to the leg brace. In some embodiments, the torso-brace socket is coupled to the torso brace.

In some embodiments, the torso brace includes two torso-brace sections that are configured to be securely coupled to one another around the torso of the subject. In some embodiments, the leg brace includes two leg-brace sections that are configured to be securely coupled to one another around the leg of the subject.

In some embodiments, the torso brace includes exactly one torso brace; the leg brace is one of exactly (only) two leg braces, and the immobilization device includes only the two leg braces; the connecting rod is one of only two connecting rods, and the immobilization device includes only the two connecting rods; and the leg-brace joint assembly is one of only two leg-brace joint assemblies, and the immobilization device includes only the two leg-brace joint assemblies, which are coupled to respective ones of the leg braces and the connecting rods.

There is further provided, in accordance with an application of the present invention, apparatus including:

a brace, which includes one or more brace sections that are configured to be securely coupled to one another around an anatomical portion of a human subject selected from the group consisting of: a torso and a limb;

a brace slide rod, which is fixed to the brace at one or more locations along the brace slide rod;

a connecting rod; and

a joint assembly, which is coupled to the connecting rod, and which includes a brace-slide-rod interface, which is slidably coupled to the brace slide rod.

In some embodiments, the joint assembly further includes a brace-slide-rod locking mechanism, which (a) when in an unlocked state, allows (i) sliding of the brace-slide-rod interface with respect to the brace slide rod and (ii) pivoting of the connecting rod with respect to the brace slide rod, and (b) when in a locked state, prevents (i) sliding of the brace-slide-rod interface with respect to the brace slide rod and (ii) pivoting of the connecting rod with respect to the brace slide rod.

In some embodiments, the joint assembly includes a ball-and-socket joint, which is coupled to the connecting rod, and which includes a socket and a ball fitted into the socket. In some embodiments, the joint assembly includes a ball-and-socket-joint locking mechanism, which (a) when in an unlocked state, allows pivoting of the connecting rod with respect to the socket, and (b) when in a locked state, prevents pivoting of the connecting rod with respect to the socket. In some embodiments, an end of the connecting rod has a characteristic selected from the group consisting of: the end of the connecting rod is permanently coupled to the leg-brace ball, such that the leg-brace joint assembly is coupled to the connecting rod; the end of the connecting rod is coupleable to the leg-brace ball, such that the leg-brace joint assembly is coupled to the connecting rod; and the end of the connecting rod is shaped so as to define the leg-brace ball, such that the leg-brace joint assembly is coupled to the connecting rod.

In some embodiments, at least a portion of the brace slide rod is curved.

In some embodiments:

the brace slide rod is a first brace slide rod, the connecting rod is a first connecting rod, the joint assembly is a first joint assembly, and the brace-slide-rod interface is a first brace-slide-rod interface, and

the apparatus further includes:
a second brace slide rod, which is fixed to the brace at one or more locations along the second brace slide rod;
a second connecting rod; and
a second joint assembly, which is coupled to the second connecting rod, and which includes a second brace-slide-rod interface, which is slidably coupled to the second brace slide rod.

In some embodiments, the brace includes a torso brace that is configured to be securely coupled around the torso, and the apparatus further includes a limb brace, which is configured to be securely coupled around a limb of the subject, and which is removably coupled to the connecting rod.

There is still further provided, in accordance with an application of the present invention, apparatus including:

a brace, which is configured to be securely coupled around an anatomical portion of a human subject selected from the group consisting of: a torso and a limb;

a rod; and

a joint assembly, which is coupled to the brace, and which includes:
a ball-and-socket joint, which includes a socket and a ball fitted into the socket;
a rod interface, which is slidably coupleable to the rod;
a rod locking mechanism, which (a) when in a rod-unlocked state, allows sliding of the rod interface with respect to the rod, and (b) when in a rod-locked state, prevents sliding of the rod interface with respect to the rod; and
a ball-and-socket-joint locking mechanism, which (a) when in a ball-and-socket-unlocked state, allows pivoting of the rod interface with respect to the brace, and (b) when in a ball-and-socket-locked state, prevents pivoting of the rod interface with respect to the brace.

In some embodiments, the brace includes a limb brace, which is configured to be securely coupled around the limb. In some embodiments, the joint assembly is a limb-brace joint assembly, which is coupled to the limb brace; the rod includes a connecting rod; and the apparatus further includes: a torso brace, which is configured to be securely coupled around the torso; and a torso-brace joint assembly, which is coupled to the torso brace and to the connecting rod. In some embodiments, the ball-and-socket joint comprises a limb ball-and-socket joint; the ball-and-socket-joint locking mechanism comprises a limb-ball-and-socket-joint locking mechanism, configured to assume limb-ball-and-socket-locked and -unlocked states; and the torso-brace joint assembly includes a torso-ball-and-socket-joint locking mechanism, which (a) when in a torso-ball-and-socket-unlocked state, allows pivoting of the connecting rod with respect to the torso brace, and (b) when in a torso-ball-and-socket-locked state, prevents pivoting of the connecting rod with respect to the torso brace.

In some embodiments, the connecting rod is shaped so as to define a non-circular cross-section perpendicular to a longitudinal axis thereof. In some embodiments, the rod interface is shaped so as to define an opening for accepting the connecting rod, the non-circular cross-section has major and minor axes perpendicular to each other, the major axis is greater than a size of the opening of the rod interface, and the minor axis is less than the size of the opening of the rod interface. In some embodiments, a perimeter of the non-circular cross-section is shaped as first and second non-contiguous portions that form respective arcs of a single circle, which first and second non-contiguous portions are separated around the perimeter by third and fourth non-contiguous portions that do not form arcs of the circle.

In some embodiments, the brace includes a torso brace, which is configured to be securely coupled around the torso. In some embodiments, the joint assembly is a torso-brace joint assembly, which is coupled to the torso brace; the rod includes a brace slide rod, which is fixed to the torso brace at one or more locations along the brace slide rod; and the joint assembly is coupled to the torso brace via the brace slide rod.

In some embodiments, the apparatus further includes: a connecting rod, which is coupled to the torso-brace joint assembly; a limb brace, which is configured to be securely coupled around the limb; and a limb-brace joint assembly, which is coupled to the limb brace and to the connecting rod. In some embodiments, the limb is a leg of the subject, the limb brace includes a leg brace, which is configured to be securely coupled around the leg, and the limb-brace joint assembly includes a leg-brace joint assembly.

In some embodiments, the rod locking mechanism is threaded. In some embodiments, the rod locking mechanism includes a screw.

In some embodiments, the ball-and-socket-joint locking mechanism is threaded.

In some embodiments, the ball-and-socket-joint locking mechanism is threaded, and having an additional locking eccentric locking bolt for further locking securing.

In some embodiments, the ball-and-socket joint includes: a socket, which is coupled directly or indirectly, to the brace; and a ball, which is fitted into the socket, and the joint assembly includes a linking member, which is shaped so as to define the ball at a first end thereof, and a post at a second end thereof, which post is coupled to the rod interface. In some embodiments, the rod interface includes a rod-interface external housing that is shaped so as to define a cylindrical threaded, internal chamber, the post is shaped so as to define an external threaded surface that is threadedly coupled to the cylindrical thread of the chamber, and rotation of the post with respect to the cylindrical threaded chamber transitions the rod locking mechanism between the rod-unlocked state and the rod-locked state.

For any of the applications described above, the ball-and-socket joint may include: a ball-and-socket external housing, that is shaped so as to define a cylindrical threaded chamber; and an internal cylindrical member, which is coupled to the brace, and which is shaped so as to define an external threaded surface that is threadedly coupled to the cylindrical threaded chamber, and rotation of the ball-and-socket external housing with respect to the internal cylindrical member transitions the ball-and-socket-joint locking mechanism between the ball-and-socket-unlocked state and the ball-and-socket-locked state.

There is additionally provided, For any of the applications described above, the ball-and-socket joint may include: a ball-and-socket external housing, that is shaped so as to define a cylindrical threaded chamber; and an internal cylindrical member, which is coupled to the brace, and which is shaped so as to define an external threaded surface that is threadedly coupled to the cylindrical threaded chamber, and rotation of the ball-and-socket external housing with respect to the internal cylindrical member maximally approximate the ball-and-socket-joint members, the internal cylindrical member further comprises an internal piston, comprising on its upper end a socket and is adopted to move up and down along the longitudinal axis of the internal cylindrical member, up and down movement is activated by an eccentric bolt located inside the piston, turning the eccentric bolt moves the piston upwards thereby locking the socket to the ball.

There is additionally provided, in accordance with an application of the present invention, apparatus including an immobilization device, which includes:

a brace system, which includes one or more braces, each of which braces includes one or more brace sections that are configured to be securely coupled to one another around an anatomical portion of a human subject selected from the group consisting of: a torso and a limb; and

a joint series assembly, which includes:
a connecting rod;
at least one first joint assembly, which is coupled to the brace system, and which includes:
   a first ball-and-socket joint, which includes a first socket and a first ball fitted into the first socket;
   a first rod interface, which is coupled to the first ball and coupleable to the connecting rod; and
   a first ball-and-socket-joint locking mechanism, which (a) when in a first ball-and-socket-unlocked state, allows pivoting of the first rod interface with respect to the brace system, and (b) when in a first ball-and-socket-locked state, prevents pivoting of the first rod interface with respect to the brace system; and
at least one second joint assembly, which is coupled to the brace system, and which includes:
   a second ball-and-socket joint, which includes a second socket and a second ball fitted into the second socket;
   a second rod interface, which is coupled to the second ball and slidably coupleable to the connecting rod;
   a rod locking mechanism, which (a) when in a rod-unlocked state, allows sliding of the second rod interface with respect to the connecting rod, and (b) when in a rod-locked state, prevents sliding of the second rod interface with respect to the connecting rod; and
   a second ball-and-socket-joint locking mechanism, which (a) when in a second ball-and-socket-unlocked state, allows pivoting of the second rod interface with respect to the brace system, and (b) when in a second ball-and-socket-locked state, prevents pivoting of the second rod interface with respect to the brace system,

wherein the at least one first joint assembly and the at least one second joint assembly are configured, when coupled together by the connecting rod and coupled to the one or more braces, to provide a frame that is configured to assume:
a frame-unlocked state when (a) the first ball-and-socket-joint locking mechanism is in the first ball-and-socket-unlocked state, (b) the rod locking mechanism is in the rod-unlocked state, and (c) the second ball-and-socket-joint locking mechanism is in the second ball-and-socket-unlocked state, in which frame-unlocked state each of the braces can be rotated and tilted to a desired anatomical position, and
a frame-locked state when (a) the first ball-and-socket-joint locking mechanism is in the first ball-and-socket-locked state, (b) the rod locking mechanism is in the rod-locked state, and (c) the second ball-and-socket-joint locking mechanism is in the second ball-and-socket-locked state, in which frame-locked state the one or more braces are locked in the desired anatomical position.

In some embodiments, an end of the connecting rod is shaped so as to define the first ball, such that the first joint assembly is coupled to the connecting rod.

There is yet additionally provided, in accordance with an application of the present invention, apparatus including an immobilization device, which includes:

a brace system, which includes one or more braces, each of which includes one or more brace sections that are configured to be securely coupled to one another around an anatomical portion of a human subject selected from the group consisting of: a torso and a limb; and

a joint series assembly, which includes:
a connecting rod having an end that is shaped so as to define a first ball;
at least one first joint assembly, which is coupled to the brace system, and which includes:
   a first ball-and-socket joint, which includes a first socket and the first ball fitted into the first socket, such that the first joint assembly is coupled to the connecting rod; and
   a first ball-and-socket-joint locking mechanism, which (a) when in a first ball-and-socket-unlocked state, allows pivoting of the connecting rod with respect to the brace system, and (b) when in a first ball-and-socket-locked state, prevents pivoting of the connecting rod with respect to the brace system; and
at least one second joint assembly, which is coupled to the brace system, and which includes:
   a second ball-and-socket joint, which includes a second socket and a second ball fitted into the second socket;
   a rod interface, which is coupled to the second ball and slidably coupleable to the connecting rod;
   a rod locking mechanism, which (a) when in a od-unlocked state, allows sliding of the rod interface with respect to the connecting rod, and (b) when in a rod-locked state, prevents sliding of the rod interface with respect to the connecting rod; and
   a second ball-and-socket-joint locking mechanism, which (a) when in a second ball-and-socket-unlocked state, allows pivoting of the rod interface with respect to the brace system, and (b) when in a second ball-and-socket-locked state, prevents pivoting of the rod interface with respect to the brace system,

wherein the at least one first joint assembly and the at least one second joint assembly are configured, when coupled together by the connecting rod and coupled to the one or more braces, to provide a frame that is configured to assume:
a frame-unlocked state when (a) the first ball-and-socket-joint locking mechanism is in the first ball-and-socket-unlocked state, (b) the rod locking mechanism is in the rod-unlocked state, and (c) the second ball-and-socket-joint locking mechanism is in the second ball-and-socket-unlocked state, in which frame-unlocked state each of the braces can be rotated and tilted to a desired anatomical position, and
a frame-locked state when (a) the first ball-and-socket-joint locking mechanism is in the first ball-and-socket-locked state, (b) the rod locking mechanism is in the rod-locked state, and (c) the second ball-and-socket-joint locking mechanism is in the second ball-and-socket-locked state, in which frame-locked state the one or more braces are locked in the desired anatomical position.

There is also provided, in accordance with an application of the present invention, a method for immobilizing a portion of a human subject, including:

securely coupling a torso brace around a torso of the subject;

securely coupling a leg brace around a leg of the subject;

providing a connecting rod;

providing a torso-brace joint assembly, which is coupled to the torso brace and the connecting rod, and which includes (a) a torso-brace ball-and-socket joint including a torso-brace socket and a torso-brace ball fitted into the socket, and (b) a torso-brace locking mechanism;

providing a leg-brace joint assembly, which is coupled to the leg brace and the connecting rod, and which includes (a) a leg-brace ball-and-socket joint including a leg-brace socket and a leg-brace ball fitted into the socket, and (b) a leg-brace locking mechanism;

transitioning the torso-brace locking mechanism from (i) an unlocked state, in which the torso-brace locking mechanism is entirely open and loose, thereby allowing pivoting of the connecting rod with respect to the torso brace, to (ii) a locked state, in which the torso-brace locking mechanism is locked and thereby prevents pivoting of the connecting rod with respect to the torso brace; and

transitioning the leg-brace locking mechanism from (i) an unlocked state, in which the leg-brace locking mechanism is completely open and loose, thereby allowing pivoting of the connecting rod with respect to the leg brace, to (ii) a locked state, in which the leg-brace locking mechanism is locked, thereby prevents pivoting of the connecting rod with respect to the leg brace.

There is further provided, in accordance with an application of the present invention, a method including:

providing (a) a brace, (b) a brace slide rod, which is fixed to the brace at one or more locations along the brace slide rod, (c) a connecting rod, and (d) a joint assembly, which is coupled to the connecting rod, and which includes a brace-slide-rod interface, which is slidably coupled to the brace slide rod; and

securely coupling the brace around an anatomical portion of a human subject selected from the group consisting of: a torso and a limb.

In some embodiments, the method further includes: providing a brace-slide-rod locking mechanism of the joint assembly; and transitioning the brace-slide-rod locking mechanism from (a) an unlocked state, in which the brace-slide-rod locking mechanism allows (i) sliding of the brace-slide-rod interface with respect to the brace slide rod and (ii) pivoting of the connecting rod with respect to the brace slide rod, to (b) a locked state, in which the brace-slide-rod locking mechanism prevents (i) sliding of the brace-slide-rod interface with respect to the brace slide rod and (ii) pivoting of the connecting rod with respect to the brace slide rod.

There is still further provided, in accordance with an application of the present invention, a method for immobilizing a portion of a human subject, including: providing at least first and second braces;

securely coupling the first and the second braces around respective anatomical portions of the subject, each selected from the group consisting of: a torso and a limb;

providing a connecting rod, which couples the first brace to the second brace?

providing a joint assembly, which is coupled to the first brace, and which includes (a) a ball-and-socket joint, which includes a socket and a ball fitted into the socket, (b) a connecting-rod interface, which is slidably coupleable to the connecting rod, (c) a connecting-rod locking mechanism, and (d) a ball-and-socket-joint locking mechanism;

transitioning the connecting-rod locking mechanism from (a) a connecting-rod-unlocked state, in which the connecting-rod locking mechanism allows sliding of the connecting-rod interface with respect to the connecting rod, to (b) a connecting-rod-locked state, in which the connecting-rod locking mechanism prevents sliding of the connecting-rod interface with respect to the connecting rod; and

transitioning the ball-and-socket-joint locking mechanism from (a) a ball-and-socket-unlocked state, in which the ball-and-socket-joint locking mechanism allows pivoting of the connecting-rod interface with respect to the first brace, to (b) a ball-and-socket-locked state, in which the ball-and-socket-joint locking mechanism prevents pivoting of the connecting-rod interface with respect to the first brace.

In some embodiments, providing the first brace includes providing a limb brace, and wherein securely coupling the first brace includes securely coupling the limb brace around the limb. In some embodiments, providing the joint assembly includes providing a limb-brace joint assembly, which is coupled to the limb brace; providing the second brace includes providing a torso brace, and a torso-brace joint assembly, which is coupled to the torso brace and to the connecting rod; and securing coupling the second brace includes securely coupling the torso brace around the torso.

In some embodiments, the ball-and-socket joint is a limb ball-and-socket joint; the ball-and-socket-joint locking mechanism is a limb-ball-and-socket-joint locking mechanism, configured to assume limb-ball-and-socket-locked and -unlocked states; providing the torso-brace joint assembly includes providing the torso-brace joint assembly including a torso-ball-and-socket-joint locking mechanism; and the method further includes transitioning the torso-ball-and-socket-joint locking mechanism from (a) a torso-ball-and-socket-unlocked state, in which the torso-ball-and-socket-joint locking mechanism allows pivoting of the connecting rod with respect to the torso brace, to (b) a torso-ball-and-socket-locked state, in which the torso-ball-and-socket-joint locking mechanism prevents pivoting of the connecting rod with respect to the torso brace.

There is additionally provided, in accordance with an application of the present invention, a method for immobilizing a portion of a human subject, including:

securely coupling one or more braces of a brace system around respective anatomical portions of the subject selected from the group consisting of: a torso and a limb;

providing a connecting rod;

providing a joint series assembly, which includes (a) at least one first joint assembly, which is coupled to the brace system, and which includes (i) a first ball-and-socket joint, which includes a first socket and a first ball fitted into the first socket, (ii) a first rod interface, which is coupled to the first ball and coupleable to the connecting rod, and (iii) a first ball-and-socket-joint locking mechanism, and (b) at least one second joint assembly, which is coupled to the brace system, and which includes (i) a second ball-and-socket joint, which includes a second socket and a second ball fitted into the second socket, (ii) a second rod interface, which is coupled to the second ball and slidably coupleable to the connecting rod, (iii) a rod locking mechanism, and (iv) a second ball-and-socket-joint locking mechanism;

coupling the at least one first joint assembly and the at least one second joint assembly together by the connecting rod so as to provide a frame;

while the frame is in a frame-unlocked state, rotating and tilting each of the braces to a desired anatomical position; and

transitioning the frame from the frame-unlocked state to a frame-locked state, in which the one or more braces are locked in the desired anatomical position, by:
transitioning the first ball-and-socket-joint locking mechanism from (a) a first ball-and-socket-unlocked state, in which the first ball-and-socket-joint locking mechanism allows pivoting of the first rod interface with respect to the brace system, to (b) a first ball-and-socket-locked state, in which the first ball-and-socket-joint locking mechanism prevents pivoting of the first rod interface with respect to the brace system,
transitioning the rod locking mechanism from (a) a rod-unlocked state, in which the rod locking mechanism allows sliding of the second rod interface with respect to the connecting rod, to (b) a rod-locked state, in which the rod locking mechanism prevents sliding of the second rod interface with respect to the connecting rod, and
transitioning the second ball-and-socket-joint locking mechanism from (a) a second ball-and-socket-unlocked state, in which the second ball-and-socket-joint locking mechanism allows pivoting of the second rod interface with respect to the brace system, to (b) a second ball-and-socket-locked state, in which the second ball-and-socket-joint locking mechanism prevents pivoting of the second rod interface with respect to the brace system.

In some embodiments, coupling the at least one first joint assembly and the at least one second joint assembly together by the connecting rod includes securing the connecting rod inside the second rod interface by coupling an end cap to an end of the connecting rod.

There is yet additionally provided, in accordance with an application of the present invention, a method for immobilizing a portion of a human subject, including:

securely coupling one or more braces of a brace system around respective anatomical portions of the subject selected from the group consisting of: a torso and a limb;

providing a connecting rod having an and that is shaped so as to define a first ball;

providing a joint series assembly, which includes (a) at least one first joint assembly, which is coupled to the brace system, and which includes (i) a first ball-and-socket joint, which includes a first socket and the first ball fitted into the first socket, such that the first joint assembly is coupled to the connecting rod, and (ii) a first ball-and-socket-joint locking mechanism, and (b) at least one second joint assembly, which is coupled to the brace system, and which includes (i) a second ball-and-socket joint, which includes a second socket and a second ball fitted into the second socket, (ii) a rod interface, which is slidably coupleable to the connecting rod, (iii) a rod locking mechanism, and (iv) a second ball-and-socket-joint locking mechanism;

coupling the at least one first joint assembly and the at least one second joint assembly together by the connecting rod so as to provide a frame;

while the frame is in a frame-unlocked state, rotating and tilting each of the braces to a desired anatomical position; and

transitioning the frame from the frame-unlocked state to a frame-locked state, in which the one or more braces are locked in the desired anatomical position, by:
transitioning the first ball-and-socket-joint locking mechanism from (a) a first ball-and-socket-unlocked state, in which the first ball-and-socket-joint locking mechanism allows pivoting of the connecting rod with respect to the brace system, to (b) a first ball-and-socket-locked state, in which the first ball-and-socket-joint locking mechanism prevents pivoting of the connecting rod with respect to the brace system,
transitioning the rod locking mechanism from (a) a rod-unlocked state, in which the rod locking mechanism allows sliding of the rod interface with respect to the connecting rod, to (b) a rod-locked state, in which the rod locking mechanism prevents sliding of the rod interface with respect to the connecting rod, and
transitioning the second ball-and-socket-joint locking mechanism from (a) a second ball-and-socket-unlocked state, in which the second ball-and-socket-joint locking mechanism allows pivoting of the rod interface with respect to the brace system, to (b) a second ball-and-socket-locked state, in which the second ball-and-socket-joint locking mechanism prevents pivoting of the rod interface with respect to the brace system.

In some embodiments, coupling the at least one first joint assembly and the at least one second joint assembly together by the connecting rod includes securing the connecting rod inside the second rod interface by coupling an end cap to an end of the connecting rod.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An immobilization device, comprising:
a torso brace configured to be positioned around a torso of a subject;
at least one leg brace each configured to be securely coupled around a leg of the subject;
at least one connecting rod;
at least one torso-brace joint assembly each coupled to the torso brace and a respective one of the at least one connecting rod, each of the at least one torso-brace joint assembly comprising (a) a torso-brace ball-and-socket joint comprising a torso-brace socket and a torso-brace ball fitted into the socket, and (b) a torso-brace locking mechanism having an unlocked state in which pivoting of the respective connecting rod with respect to the torso brace is possible, and a locked state in which pivoting of the respective connecting rod with respect to the torso brace is prevented; and
at least one leg-brace joint assembly, each coupled to a respective one of the at least one leg brace and a respective one of the at least one connecting rod, each of the at least one leg-brace joint assembly comprising (a) a leg-brace ball-and-socket joint comprising a leg-brace socket and a leg-brace ball fitted into the socket, and (b) a leg-brace locking mechanism having an unlocked state in which pivoting of the respective connecting rod with respect to the respective leg brace is possible, and a locked state in which pivoting of the respective connecting rod with respect to the respective leg brace is prevented.

2. The device of claim 1, wherein an end of the at least one connecting rod is permanently coupled to the torso-brace ball.

3. The device of claim 1, wherein an end of the at least one connecting rod is selectively coupled to the torso-brace ball, such that the at least one torso-brace joint assembly is removably coupled to the at least one connecting rod.

4. The device of claim 1, wherein an end of the at least one connecting rod is shaped so as to define the torso-brace ball.

5. The device of claim 1, wherein an end of the at least one connecting rod is permanently coupled to the leg-brace ball.

6. The device of claim 1, wherein an end of the at least one connecting rod is selectively coupled to the leg-brace ball, such that the at least one leg-brace joint assembly is removably coupled to the at least one connecting rod.

7. The device of claim 1, wherein an end of the at least one connecting rod is shaped so as to define the leg-brace ball.

8. The device of claim 1, wherein the leg-brace socket is coupled to the leg brace.

9. The device of claim 1, wherein the torso-brace socket is coupled to the torso brace.

10. An apparatus comprising:
at least one brace configured to be securely coupled around an anatomical portion of a human subject selected from a group consisting of: a torso and a limb;
at least one rod; and
at least one joint assembly coupled to the at least one brace, each of the at least one joint assembly comprising:
a ball-and-socket joint which comprises a socket and a ball fitted into the socket;
a rod interface slidably and selectively coupled to the at least one rod;
a rod locking mechanism having a rod-unlocked state in which sliding of the rod interface with respect to the rod is possible and a rod-locked state in which sliding of the rod interface with respect to the rod is prevented; and
a ball-and-socket-joint locking mechanism having a ball-and-socket-unlocked state in which pivoting of the rod interface with respect to the brace is possible and a ball-and-socket-locked state in which pivoting of the rod interface with respect to the brace is prevented.

11. The apparatus of claim 10, wherein the anatomical portion is a limb such that the at least one brace comprises a limb brace, and the joint assembly comprises a limb-brace joint assembly coupled to the limb brace, wherein the at least one rod comprises a connecting rod, and wherein the apparatus further comprises:
a torso brace configured to be securely coupled around the torso of the subject; and
a torso-brace joint assembly coupled to the torso brace and to the connecting rod;
wherein the ball-and-socket joint comprises a limb ball-and-socket joint,
wherein the ball-and-socket-joint locking mechanism comprises a limb-ball-and-socket-joint locking mechanism, configured to assume limb-ball-and-socket-locked and unlocked states, and
wherein the torso-brace joint assembly comprises a torso-ball-and-socket-joint locking mechanism having a torso-ball-and-socket-unlocked state in which pivoting of the connecting rod with respect to the torso brace is possible, and a torso-ball-and-socket-locked state in which pivoting of the connecting rod with respect to the torso brace is prevented.

12. The apparatus of claim 10, wherein the rod locking mechanism is threaded.

13. The apparatus of claim 12, wherein the threading of the rod locking mechanism is provided by a screw.

14. The apparatus of claim 10, wherein the ball-and-socket joint comprises:
a ball-and-socket external housing shaped to define a cylindrical threaded, internal chamber; and
an internal cylindrical member which is coupled to the at least one brace, and which is shaped to define an external threaded surface that is threadedly coupled to the cylindrical thread of the chamber, and wherein rotation of the ball-and-socket external housing with respect to the internal cylindrical member transitions the ball-and-socket-joint locking mechanism between the ball-and-socket-unlocked state and the ball-and-socket-locked state.

15. The apparatus of claim 10, wherein the ball-and-socket joint comprises:
a ball-and-socket external housing shaped to define a cylindrical threaded, internal chamber; and
an internal base member coupled to the brace and shaped to define an external threaded surface that is threadedly coupled to the cylindrical thread of the chamber, and wherein the internal base member further contains an internal piston which comprises a socket on one end and is adapted to move up and down along a longitudinal axis of the internal base member, up and down movement being activated by an eccentric bolt arranged at least partly inside the internal piston which, when turned in one direction, moves the internal piston upwards thereby locking the socket to the ball.

16. An immobilization device, comprising:
a brace system comprising at least one brace, each of the at least one brace comprising one or more brace sections that are configured to be securely coupled to one another around an anatomical portion of a subject selected from the group consisting of: a torso and a limb; and
at least one joint series assembly each comprising:
at least one connecting rod;
at least one first joint assembly coupled to the brace system, each of the at least one first joint assembly comprising:
a first ball-and-socket joint which comprises a first socket and a first ball fitted into the first socket;
a first rod interface coupled to the first ball and selectively coupled to a respective one of the at least one connecting rod; and
a first ball-and-socket-joint locking mechanism having a first ball-and-socket-unlocked state in which pivoting of the first rod interface with respect to the brace system is possible, and a first ball-and-socket-locked state in which pivoting of the first rod interface with respect to the brace system is prevented; and
at least one second joint assembly, which is coupled to the brace system, each of the at least one second joint assembly comprising:
a second ball-and-socket joint which comprises a second socket and a second ball fitted into the second socket;
a second rod interface coupled to the second ball and slidably and selectively coupled to a respective one of the at least one connecting rod;
a rod locking mechanism having a rod-unlocked state in which sliding of the second rod interface with respect to the connecting rod is possible, and a rod-locked state in which sliding of the second rod interface with respect to the connecting rod is prevented; and
a second ball-and-socket-joint locking mechanism having a second ball-and-socket-unlocked state in which pivoting of the second rod interface with respect to the brace system is possible and a second ball-and-socket-locked state in which pivoting of the second rod interface with respect to the brace system is prevented, wherein the at least one first joint assembly and the at least one second joint assembly are configured, when coupled together by the at least one connecting rod and coupled to the at least one brace, to provide a frame that is configured to assume:
a frame-unlocked state when (a) the first ball-and-socket-joint locking mechanism is in the first ball-and-socket-unlocked state, (b) the rod locking mechanism is in the rod-unlocked state, and (c) the second ball-and-socket-joint locking mechanism is in the second ball-and-socket-unlocked state, wherein, in the frame-unlocked state, each of the at least one brace is rotatable and tiltable to a desired anatomical position, and
a frame-locked state when (a) the first ball-and-socket-joint locking mechanism is in the first ball-and-socket-locked state, (b) the rod locking mechanism is in the rod-locked state, and (c) the second ball-and-socket-joint locking mechanism is in the second ball-and-socket-locked state, wherein, in the frame-locked state, each of the at least one brace is locked in the desired anatomical position.

17. The device of claim 16, wherein an end of the at least one connecting rod is shaped to define the first ball.

18. The device of claim 16, wherein the ball-and-socket joint comprises:
a ball-and-socket external housing shaped to define a cylindrical threaded, internal chamber; and
an internal base member coupled to the brace and shaped to define an external threaded surface that is threadedly coupled to the cylindrical thread of the chamber, and wherein the internal base member further contains an internal piston which comprises a socket on one end and is adapted to move up and down along a longitudinal axis of the internal base member, up and down movement being activated by an eccentric bolt which by turning the eccentric bolt moves the internal piston upwards thereby locking the socket to the ball.

19. An immobilization device, comprising:
a brace system comprising at least one brace, each of the at least one brace comprising at least one brace section configured for positioning around an anatomical portion of a human subject selected from the group consisting of: a torso and a limb; and
at least one joint series assembly each comprising:
at least one connecting rod having an end shaped to define a first ball;
at least one first joint assembly coupled to the brace system, and which comprises:
a first ball-and-socket joint which comprises a first socket and the first ball fitted into the first socket, such that the first joint assembly is coupled to the connecting rod; and
a first ball-and-socket-joint locking mechanism having a first ball-and-socket-unlocked state in which pivoting of the connecting rod with respect to the brace system is possible, and a first ball-and-socket-locked state in which pivoting of the connecting rod with respect to the brace system is prevented; and
at least one second joint assembly coupled to the brace system, each of the at least one second joint assembly comprising:
a second ball-and-socket joint which comprises a second socket and a second ball fitted into the second socket;
a rod interface coupled to the second ball and slidably and selectively coupled to the connecting rod;
a rod locking mechanism having a rod-unlocked state in which sliding of the rod interface with respect to the connecting rod is possible, and a rod-locked state in which sliding of the rod interface with respect to the connecting rod is prevented; and
a second ball-and-socket-joint locking mechanism having a second ball-and-socket-unlocked state in which pivoting of the rod interface with respect to the brace system is possible, and a second ball-and-socket-locked state in which pivoting of the rod interface with respect to the brace system is prevented,
wherein the at least one first joint assembly and the at least one second joint assembly are configured, when coupled together by the at least one connecting rod and coupled to the at least one brace, to provide a frame that is configured to assume:
a frame-unlocked state when (a) the first ball-and-socket-joint locking mechanism is in the first ball-and-socket-unlocked state, (b) the rod locking mechanism is in the rod-unlocked state, and (c) the second ball-and-socket-joint locking mechanism is in the second ball-and-socket-unlocked state, wherein in the frame-unlocked state, each of the at least one brace is rotatable and tiltable to a desired anatomical position, and
a frame-locked state when (a) the first ball-and-socket-joint locking mechanism is in the first ball-and-socket-locked state, (b) the rod locking mechanism is in the rod-locked state, and (c) the second ball-and-socket-joint locking mechanism is in the second ball-and-socket-locked state, wherein in the frame-locked state, the at least one brace is locked in the desired anatomical position.

20. The device of claim 19, wherein the ball-and-socket joint comprises:
a ball-and-socket external housing shaped to define a cylindrical threaded, internal chamber; and
an internal base member coupled to the brace and shaped to define an external threaded surface that is threadedly coupled to the cylindrical thread of the chamber, and wherein the internal base member further contains an internal piston which comprises a socket on one end and is adapted to move up and down along a longitudinal axis of the internal base member, up and down movement being activated by an eccentric bolt arranged at least partly inside the internal piston which, when turned in one direction, moves the internal piston upwards thereby locking the socket to the ball.
